(19) United States
(12) Patent Application Publication
(10) Pub. No.: US 2004/0023214 A1
(43) Pub. Date: Feb. 5, 2004

(54) Human PHKA1-related gene variant associated with cancers

(76) Inventor: Ken-Shwo Dai, Hsinchu (TW)

Correspondence Address: Ladas & Parry, 26 West 61 Street, New York, NY, 10023, US

(21) Appl. No.: 10/102,553

(22) Filed: Aug. 2, 2002

(51) Int. Cl. C12Q 1/68; G01N 33/574; C07H 21/04; C12N 9/12; C12P 21/02; C12N 5/06

(52) U.S. Cl. 435/6; 435/7.23; 435/194; 435/69.1; 435/320.1; 435/325; 530/388.26; 536/23.2

(57) ABSTRACT

The invention relates to the nucleic acid and polypeptide of a novel human PHKA1-related gene variant.

The invention also provides the process for producing the polypeptide of the variant.

The invention further provides the use of the nucleic acid and polypeptide of the gene variant in diagnosing diseases, in particular, cancers.

### FIELD OF THE INVENTION

The invention relates to the nucleic acid a novel human PHKA1-related gene variant and the polypeptide encoded thereby, the preparation process thereof, and the uses of the same in diagnosing diseases associated with the deficiency of PHKA1 gene, in particular, cancers.

### BACKGROUND OF THE INVENTION

Lung cancer is one of the major causers of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J. Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggested that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes localized on chromosome Xq, a region shown to be associated with the development of lung cancers (Levin et al. (1995) Genes Chromosomes Cancer 13:175-85; Sundareshan and Augustus, (1996) Cancer Genet Cytogenet 91:53-60). PHKA1 is the α-subunit of phosphorylase kinase, an enzyme involved in glycogenolysis (Brushia and Walsh, (1999) Front Biosci 4:D618-41), which was mapped on this chromosome Xq12-q13 (Francke et al. (1989) Am J Hum Genet 45:276-82). This chromosomal assignment raises a possibility that PHKA1 may have a role in the tumorigenic process of lung cancers. In supporting this, glycogen level has been shown to be associated with human lung carcinoma (Yano et al. (1996) Cancer Lett 110:29-34). Thus, the discovery of gene variants of PHKA1 may be important targets for diagnostic markers of diseases.

### SUMMARY OF THE INVENTION

The present invention provides a PHKA 1-related gene variant and the polypeptide encoded thereby as well as the fragment thereof. The nucleotide sequence of the gene variant and polypeptide encoded thereby can be used for the diagnosis of diseases associated with the deficiency of PHKA1 gene, in particular, cancers.

The invention further provides an expression vector and host cell for expressing the polypeptide of the invention.

The invention further provides a method for producing the polypeptide of the invention.

The invention further provides an antibody specifically binding to the polypeptide of the invention.

The invention also provides methods for diagnosing diseases associated with the deficiency of PHKA1 gene, in particular, cancers.

### BRIEF DESCRIPTION OF THE DRAWINGS

FIG. 1 shows the nucleic acid sequence of PHKA1V (SEQ ID NO:1) and amino acid sequence encoded thereby (SEQ ID NO:2).

FIG. 2 shows the nucleotide sequence alignment between the human PHKA1 gene and PHKA1V.

FIG. 3 shows the amino acid sequence alignment between the human PHKA1 protein and PHKA1V.

### DETAILED DESCRIPTION OF THE INVENTION

According to the present invention, all technical and scientific terms used have the same meanings as commonly understood by persons skilled in the art.

The term “antibody” used herein denotes intact molecules (a polypeptide or group of polypeptides) as well as fragments thereof, such as Fab, R(ab′)₂, and Fv fragments, which are capable of binding the epitopic determinant. Antibodies are produced by specialized B cells after stimulation by an antigen. Structurally, antibody consists of four subunits including two heavy chains and two light chains. The internal surface shape and charge distribution of the antibody binding domain is complementary to the features of an antigen. Thus, antibody can specifically act against the antigen in an immune response.

The term “base pair (bp)” used herein denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402)” used herein denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence against a nucleotide sequence database;

(2) BLASTP compares an amino acid query sequence against a protein sequence database;

(3) BLASTX compares the six-frame conceptual translation products of a query nucleotide sequence against a protein sequence database;

(4) TBLASTN compares a query protein sequence against a nucleotide sequence database translated in all six reading frames; and

(5) TBLASTX compares the six-frame translations of a nucleotide query sequence against the six-frame translations of a nucleotide sequence database.

The term “cDNA” used herein denotes nucleic acids that synthesized from a mRNA template using reverse transcriptase.

The term “cDNA library” used herein denotes a library composed of complementary DNAs which are reverse-transcribed from mRNAs.

The term “complement” used herein denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion” used herein denotes a removal of a portion of one or more amino acid residues/nucleotides from a gene.

The term “expressed sequence tags (ESTs)” used herein denotes short (200 to 500 base pairs) nucleotide sequence that derives from either 5′ or 3′ end of a cDNA.

The term “expression vector” used herein denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell” used herein denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition” used herein denotes the addition of a portion of one or more amino acid residues/nucleotides to a gene.

The term “in silico” used herein denotes a process of using computational methods (e.g., BLAST) to analyze DNA sequences.

The term “polymerase chain reaction (PCR)” used herein denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide” used herein denotes a sequence of amino acids in a specific order that can be encoded by a gene or by a recombinant DNA. It can also be chemically synthesized.

The term “nucleic acid sequence” or “polynucleotide” used herein denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR)” used herein denotes a process which transcribes mRNA to complementary DNA strand using reverse transcriptase followed by polymerase chain reaction to amplify the specific fragment of DNA sequences.

The term “transformation” used herein denotes a process describing the uptake, incorporation, and expression of exogenous DNA by prokaryotic host cells.

The term “transfection” used herein a process describing the uptake, incorporation, and expression of exogenous DNA by eukaryotic host cells.

The term “variant” used herein denotes a fragment of sequence (nucleotide or amino acid) inserted or deleted by one or more nucleotides/amino acids.

The present invention in the first aspect provides the polypeptide of a novel human PHKA1-related gene variant and the fragments thereof, as well as the nucleic acid sequences encoding the same.

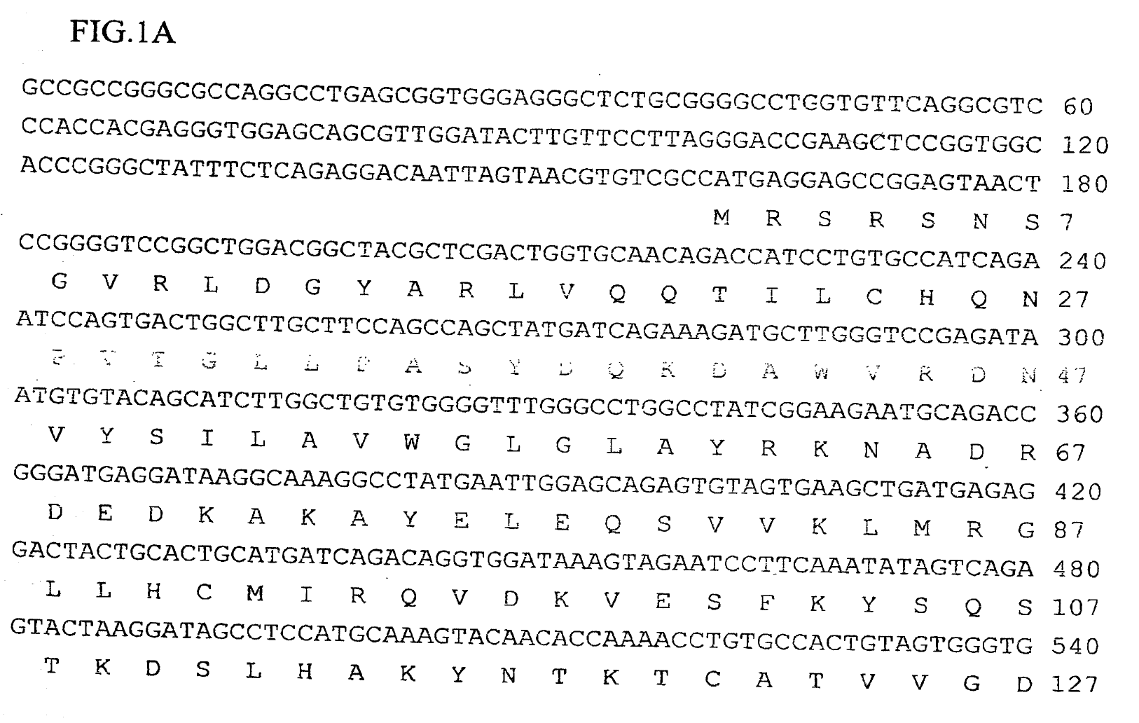

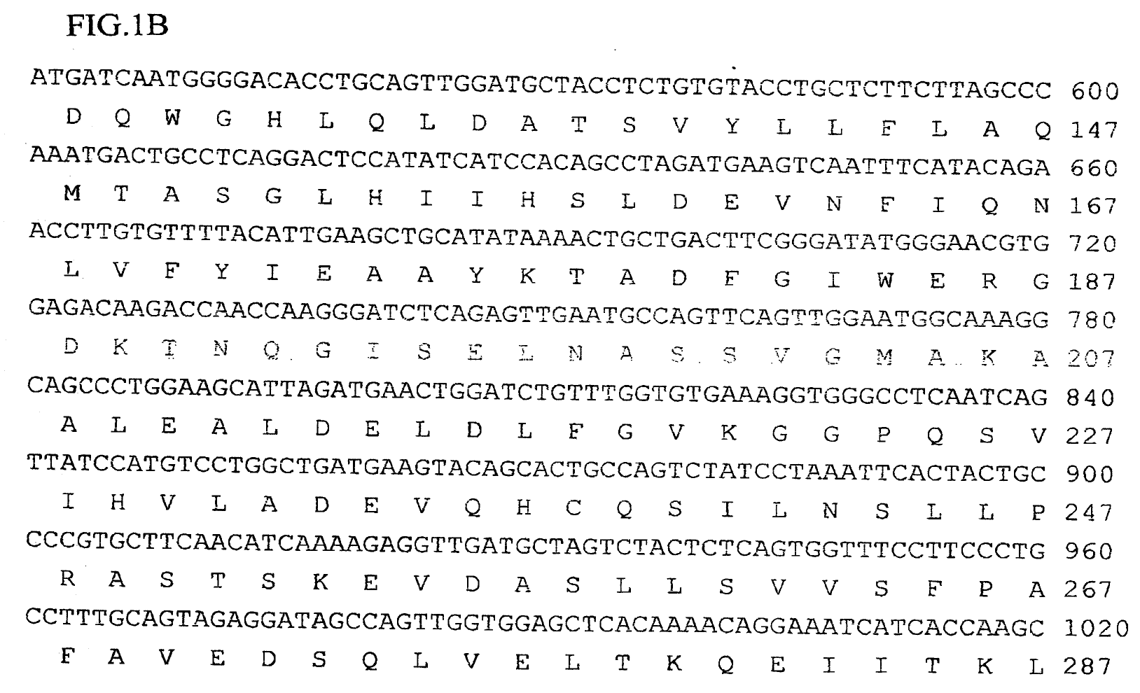

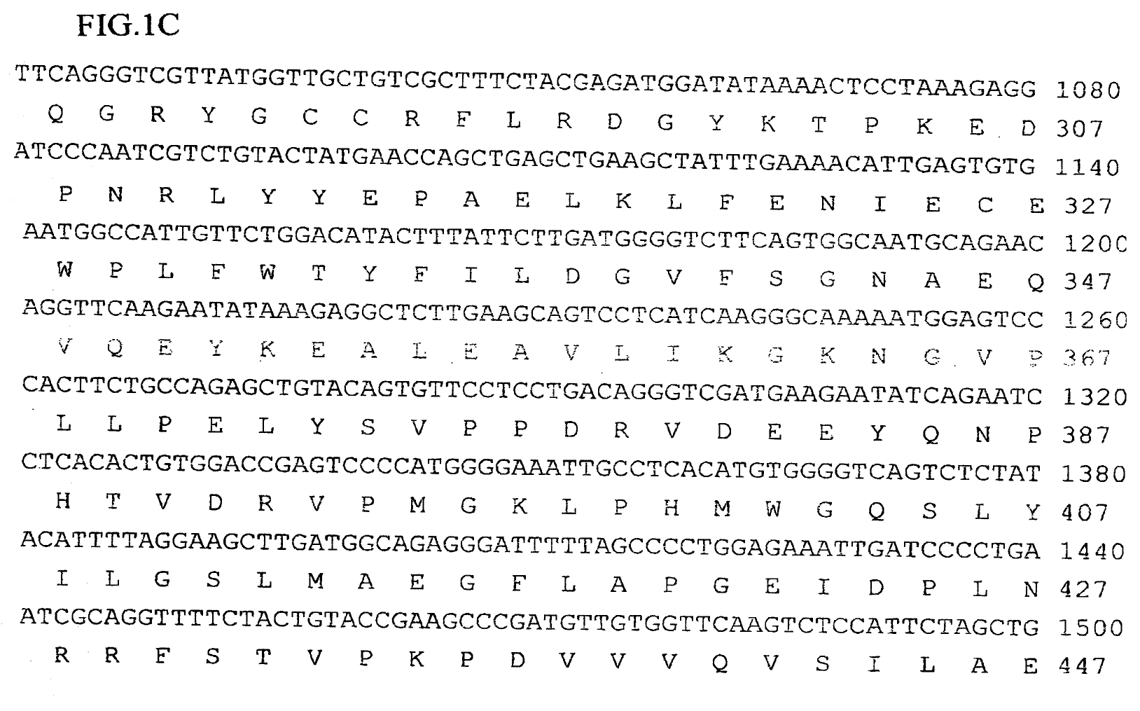

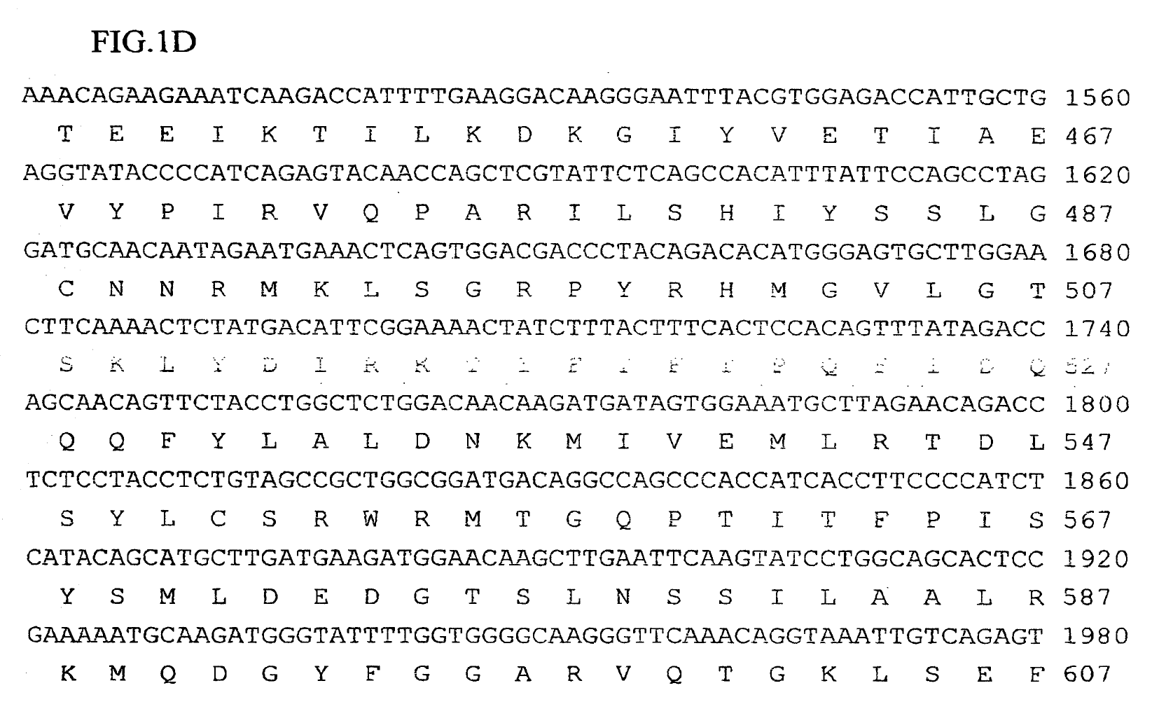

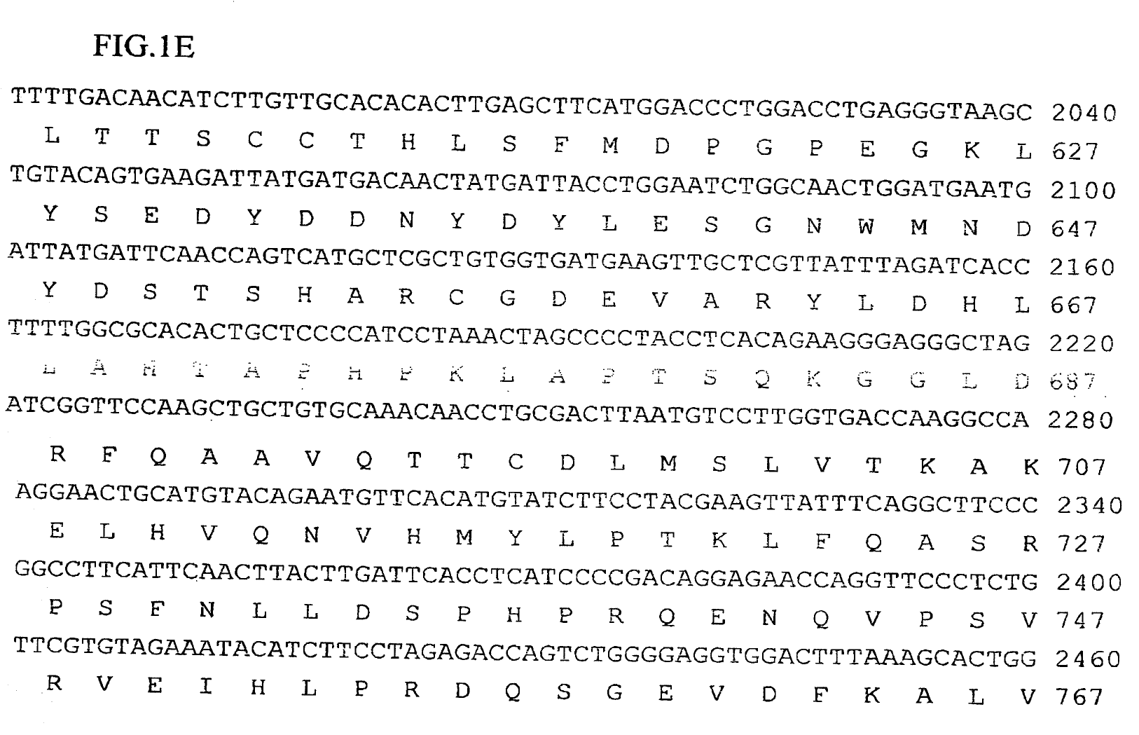

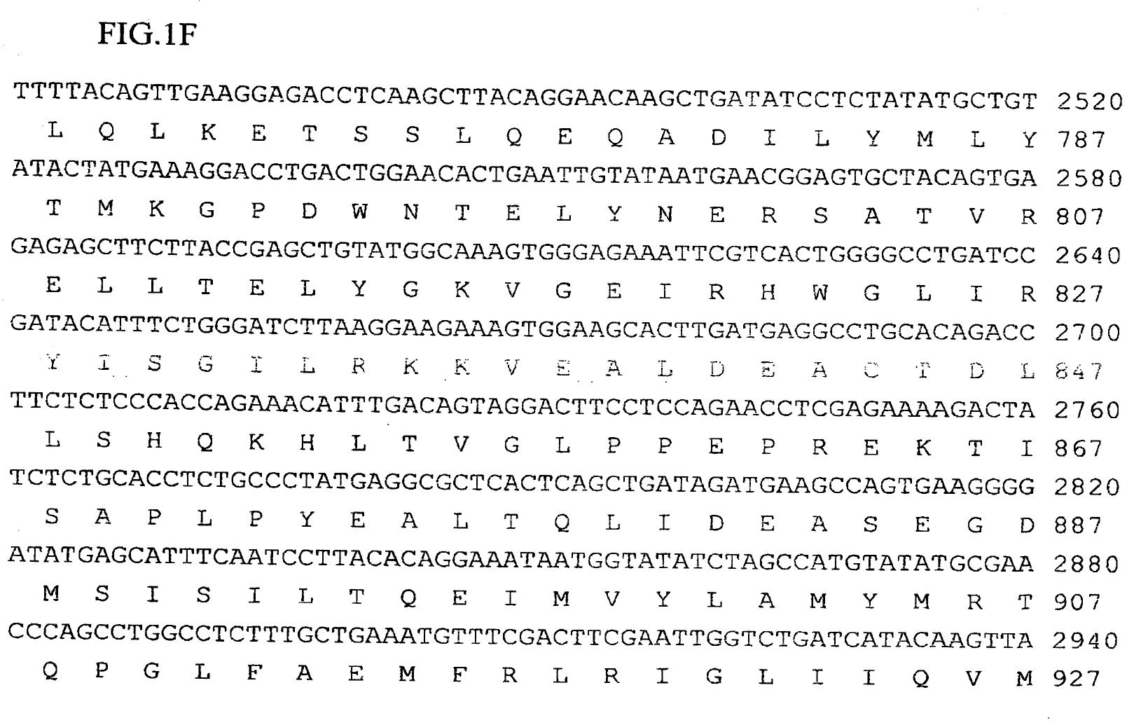

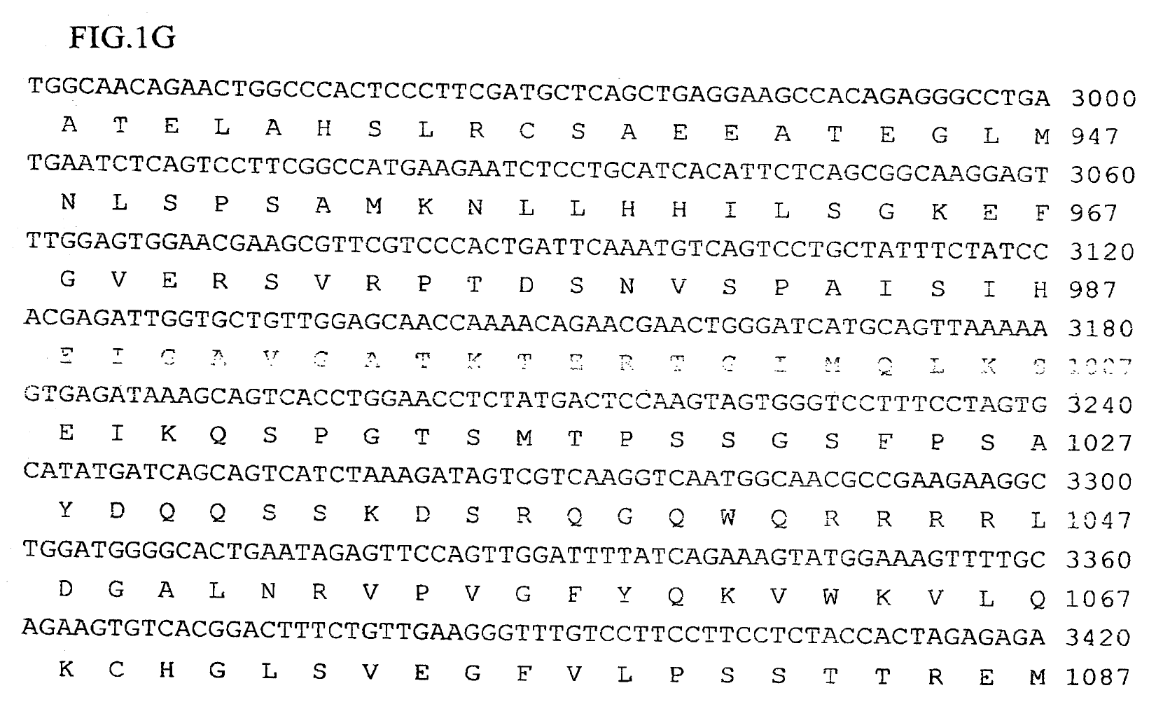

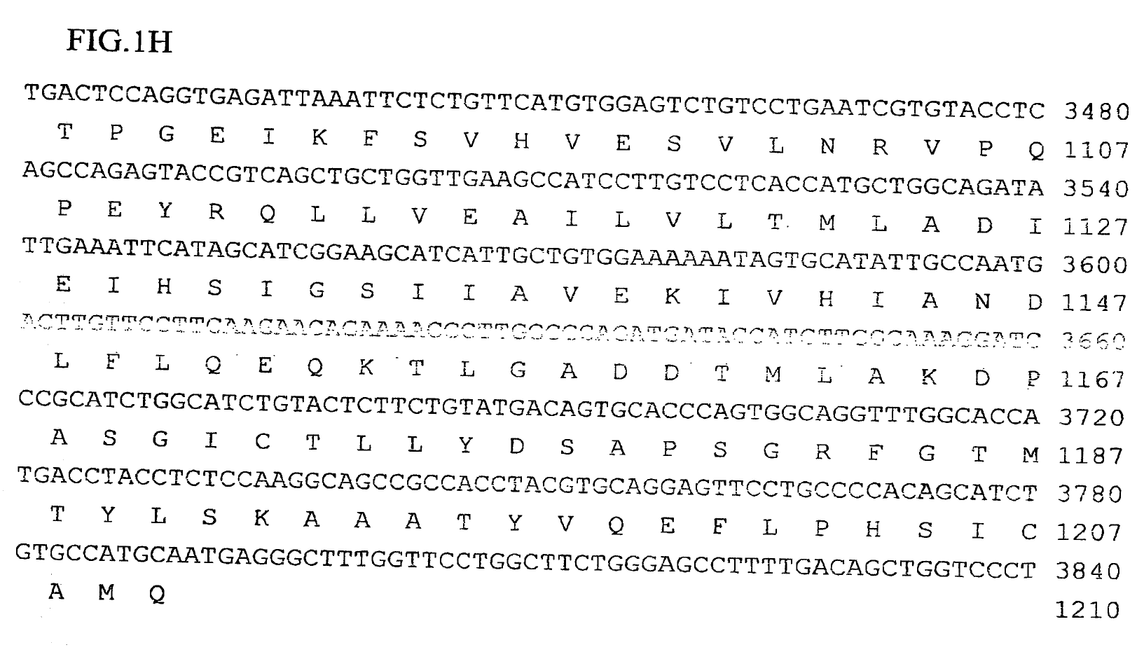

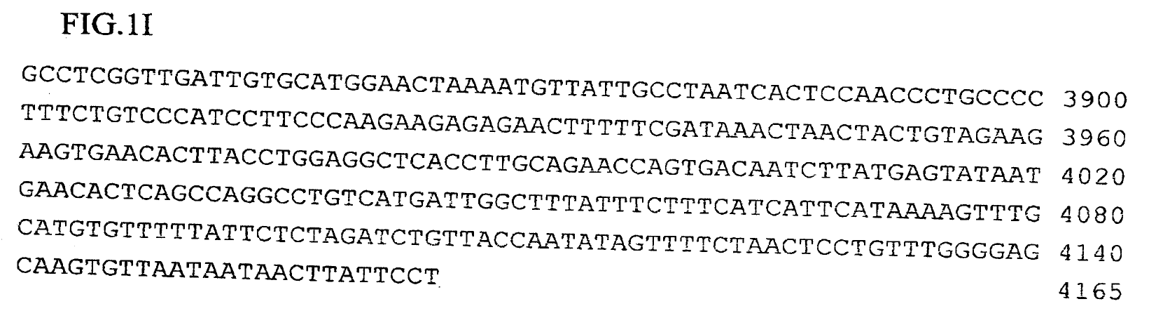

According to the present invention, human PHKA1 cDNA sequence was used to query the human lung EST databases (a normal lung, a large cell lung cancer, a squamous cell lung cancer and a small cell lung cancer) using BLAST program to search for PHKA1-related gene variants. Three ESTs showing similarity to PHKA1 were each identified from the large cell lung cancer, the squamous cell lung cancer and the SCLC databases. Their corresponding cDNA clones were found to be identical after sequencing and named PHKA1V (PHKA1 variant). FIG. 1 shows the nucleic acid sequence of PHKA1V (SEQ ID NO:1) and the amino acid sequence encoded thereby (SEQ ID NO:2).

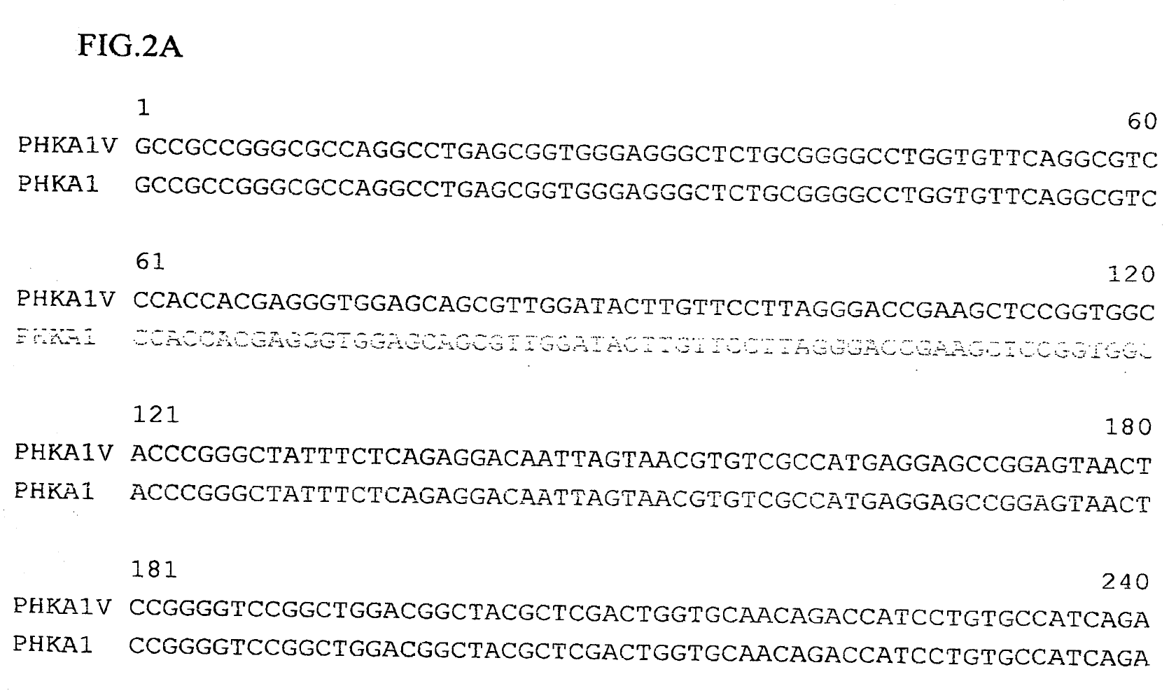

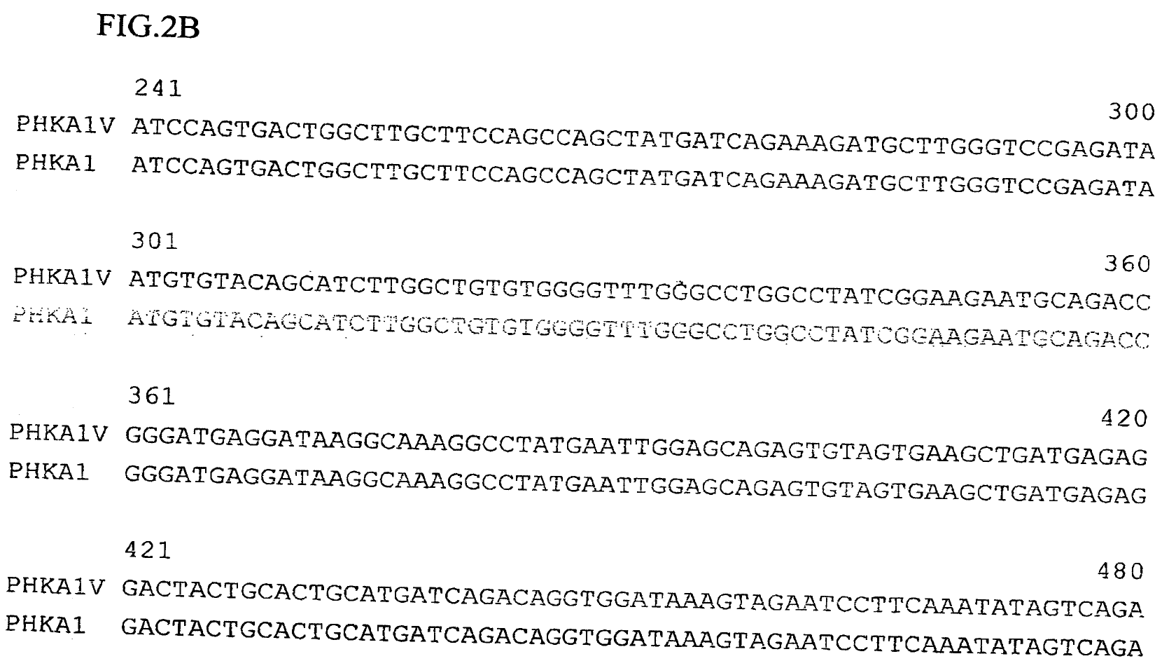

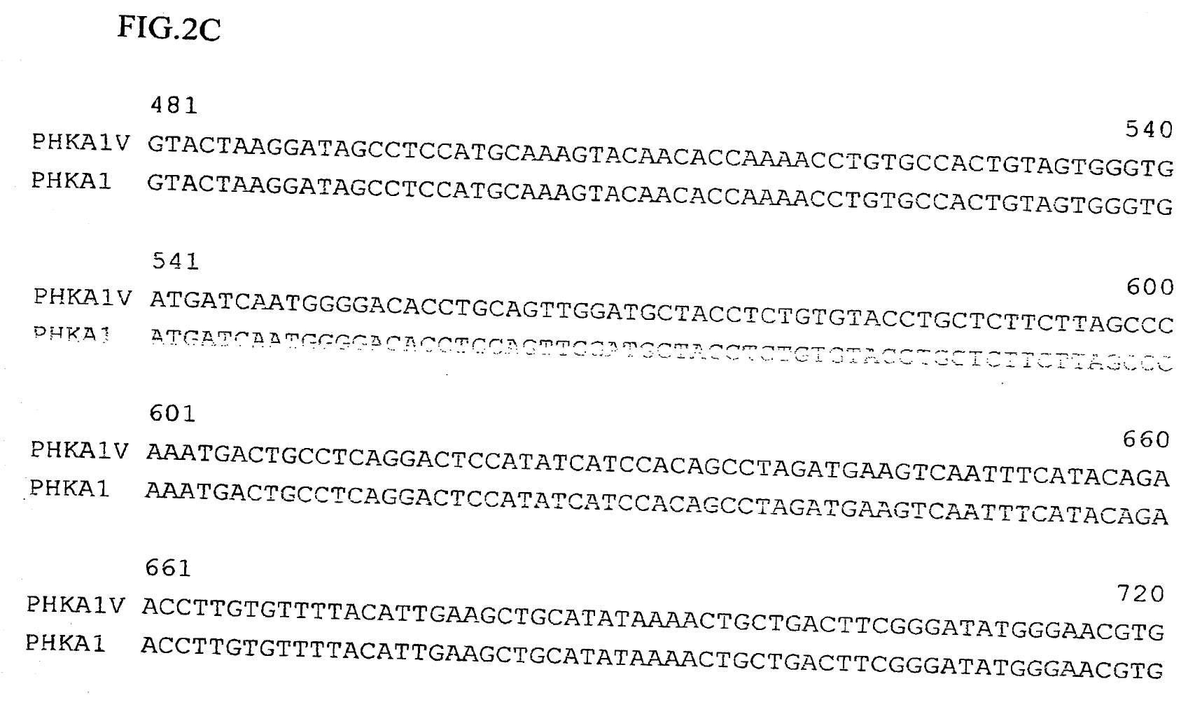

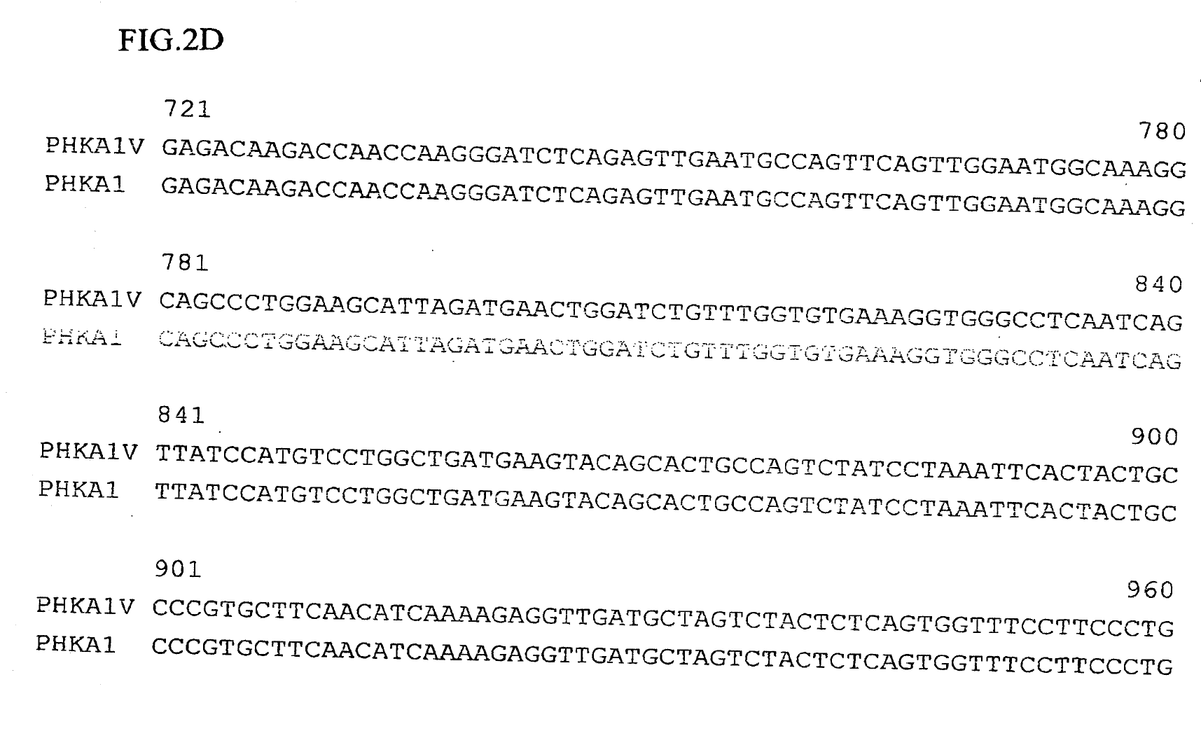

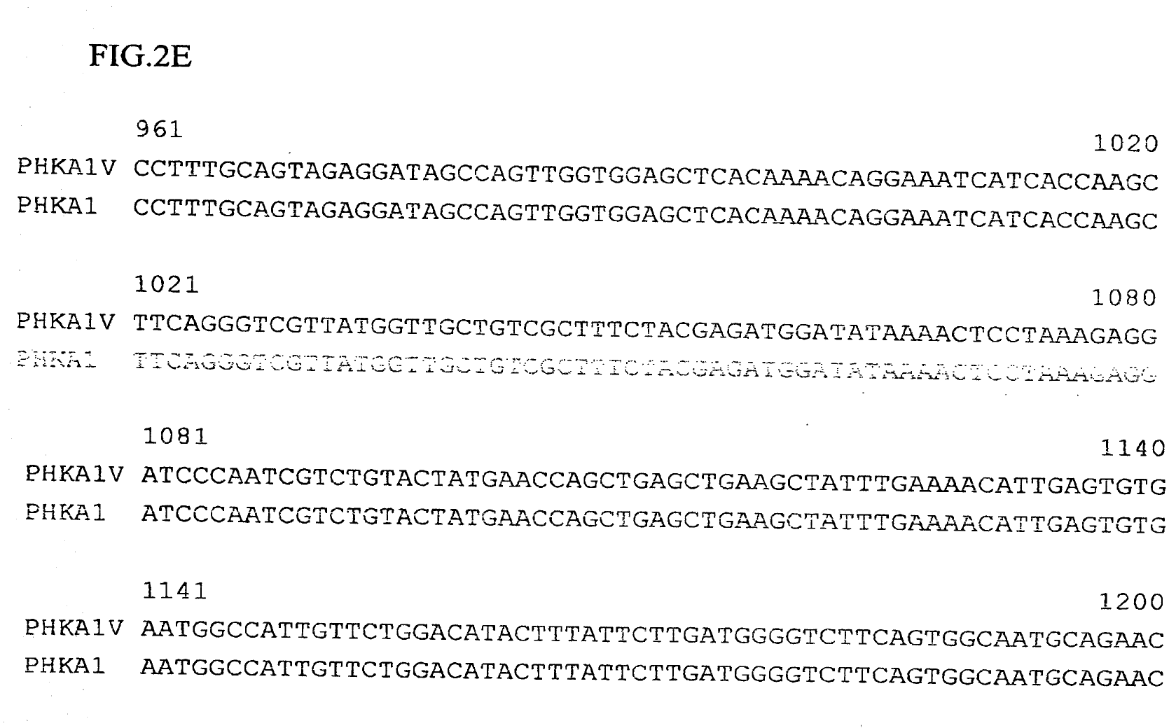

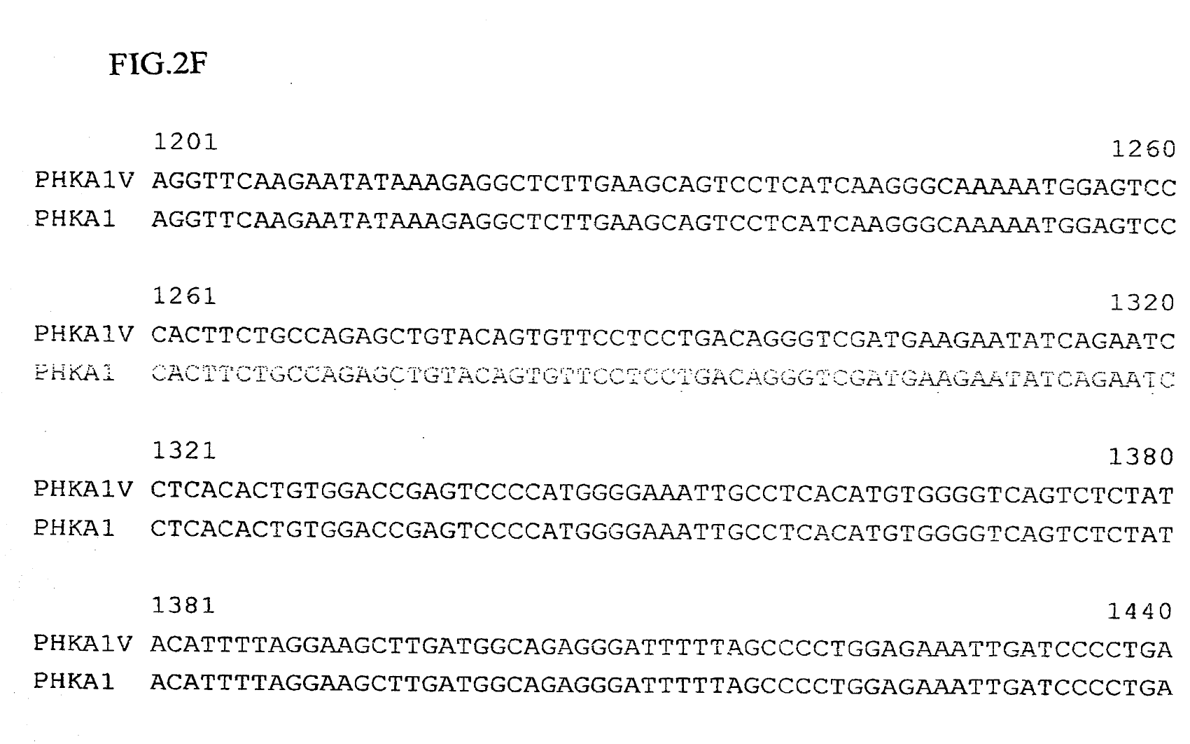

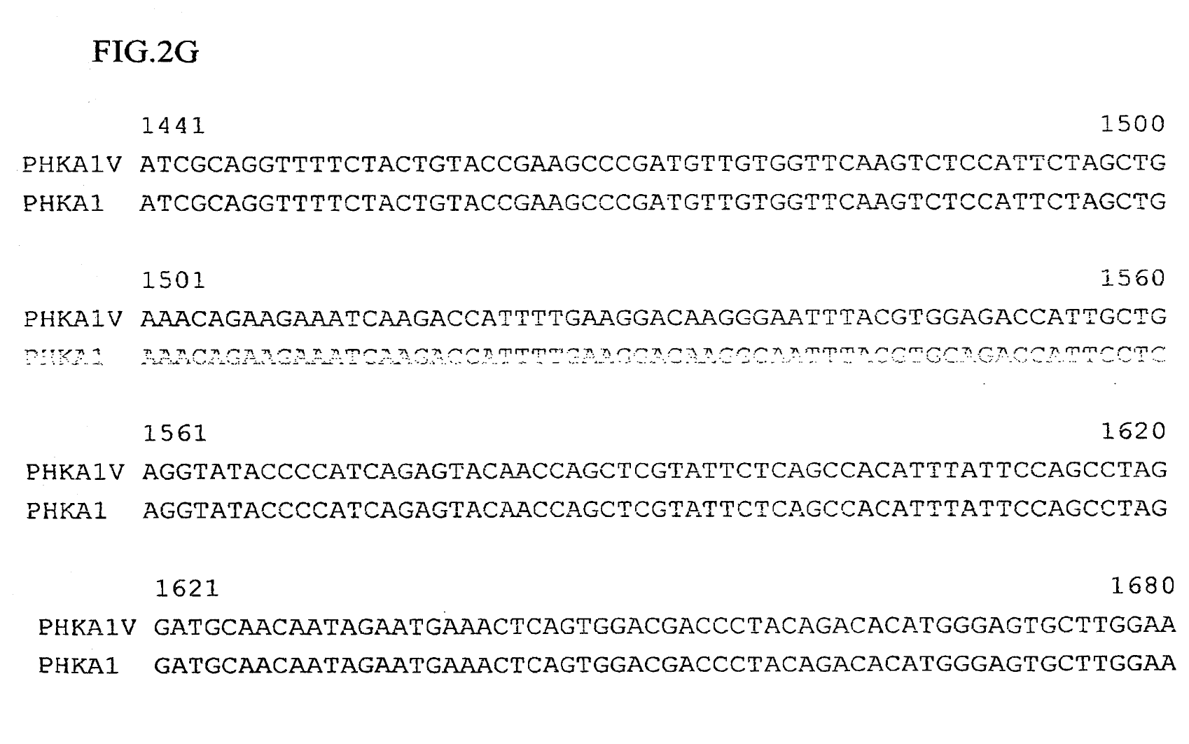

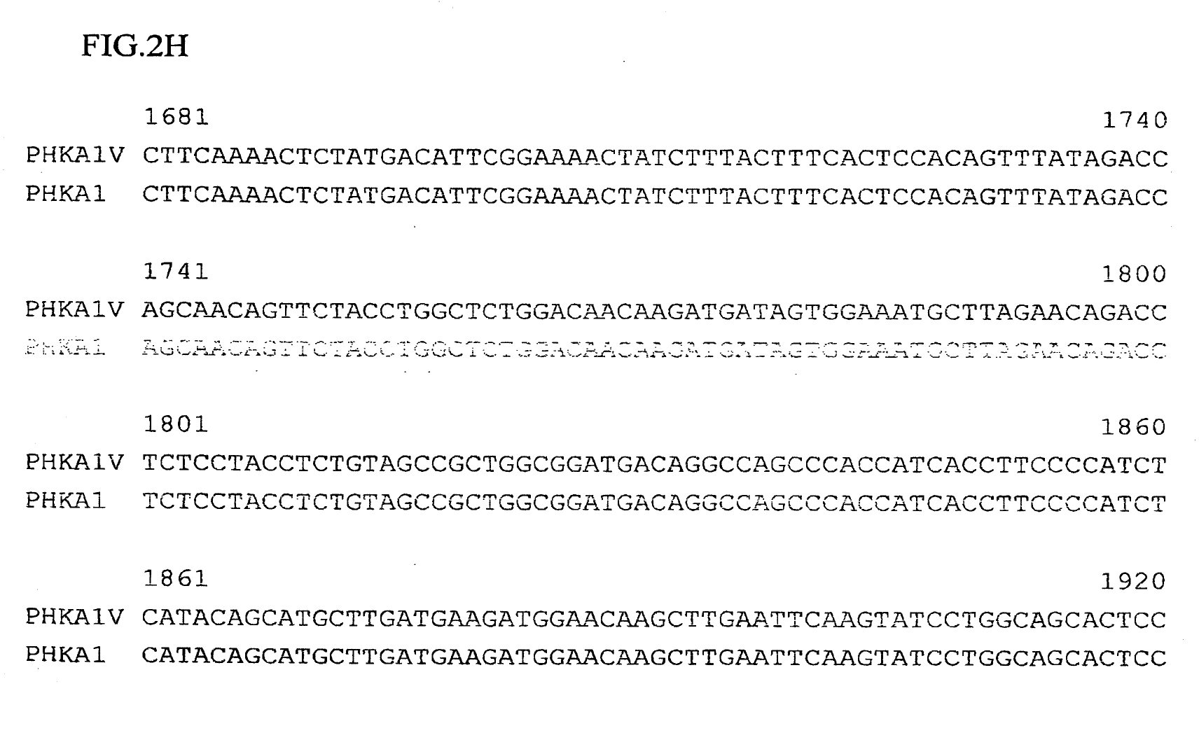

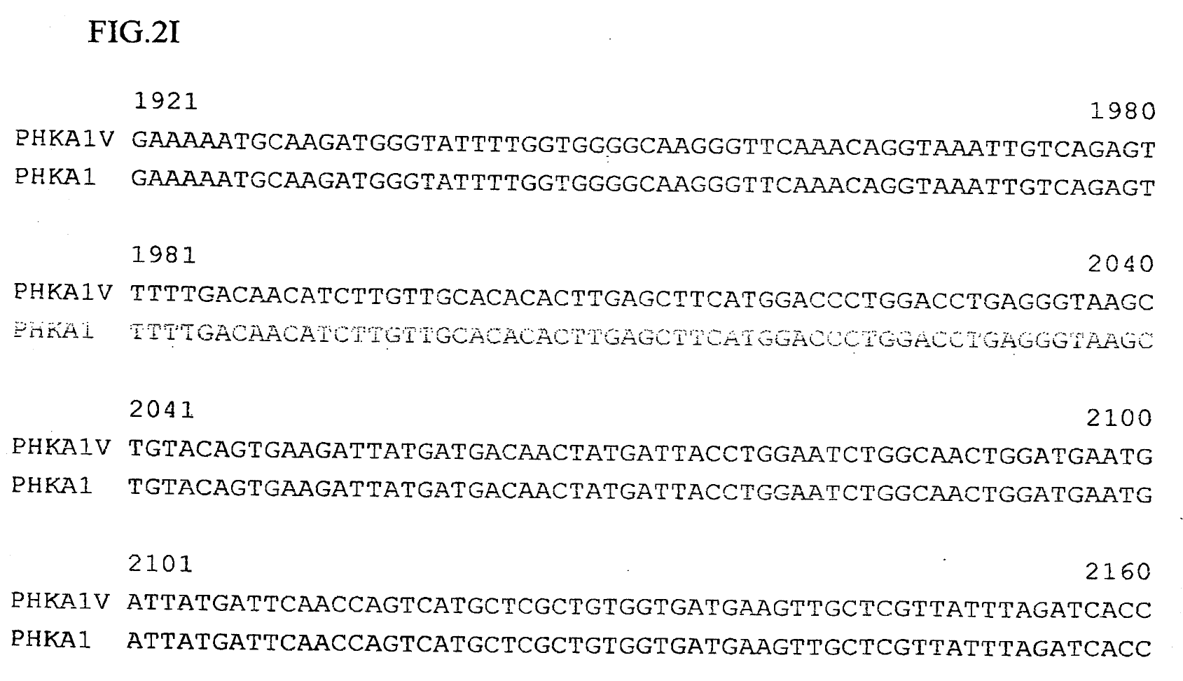

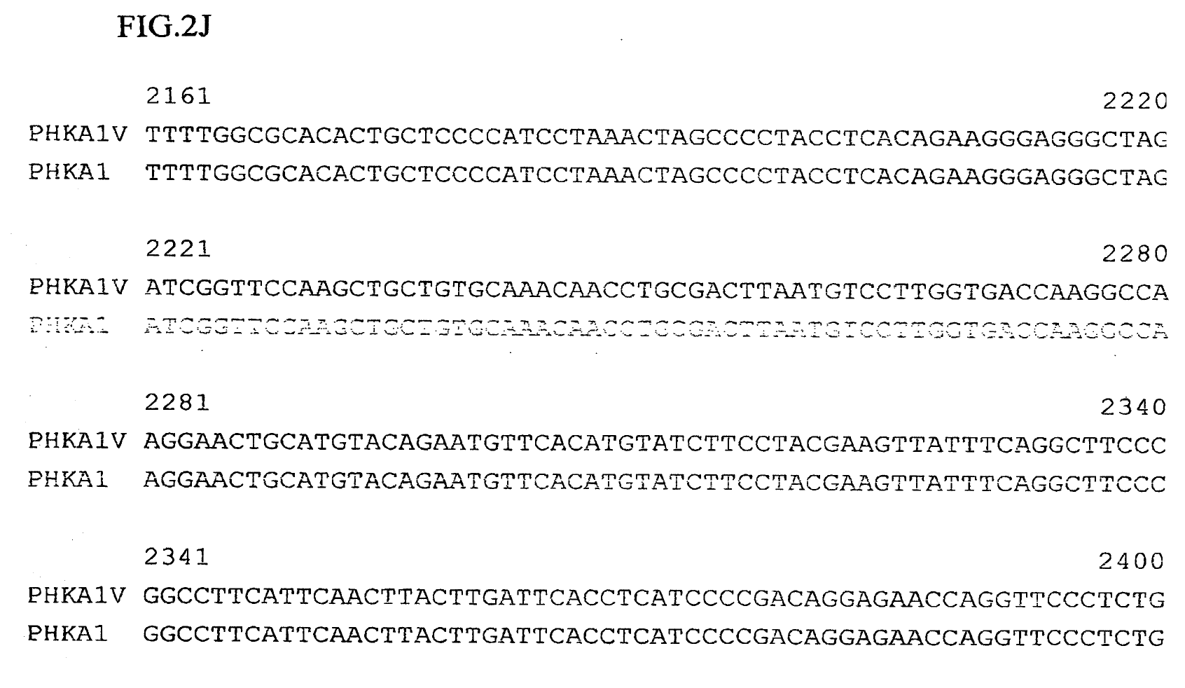

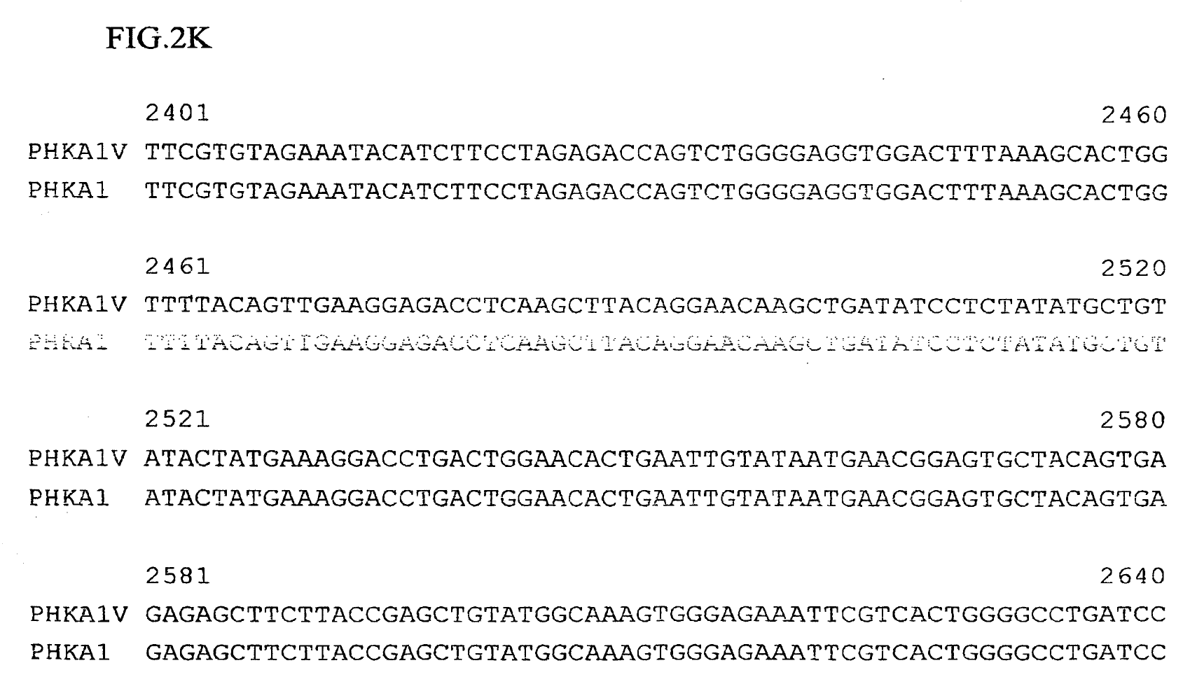

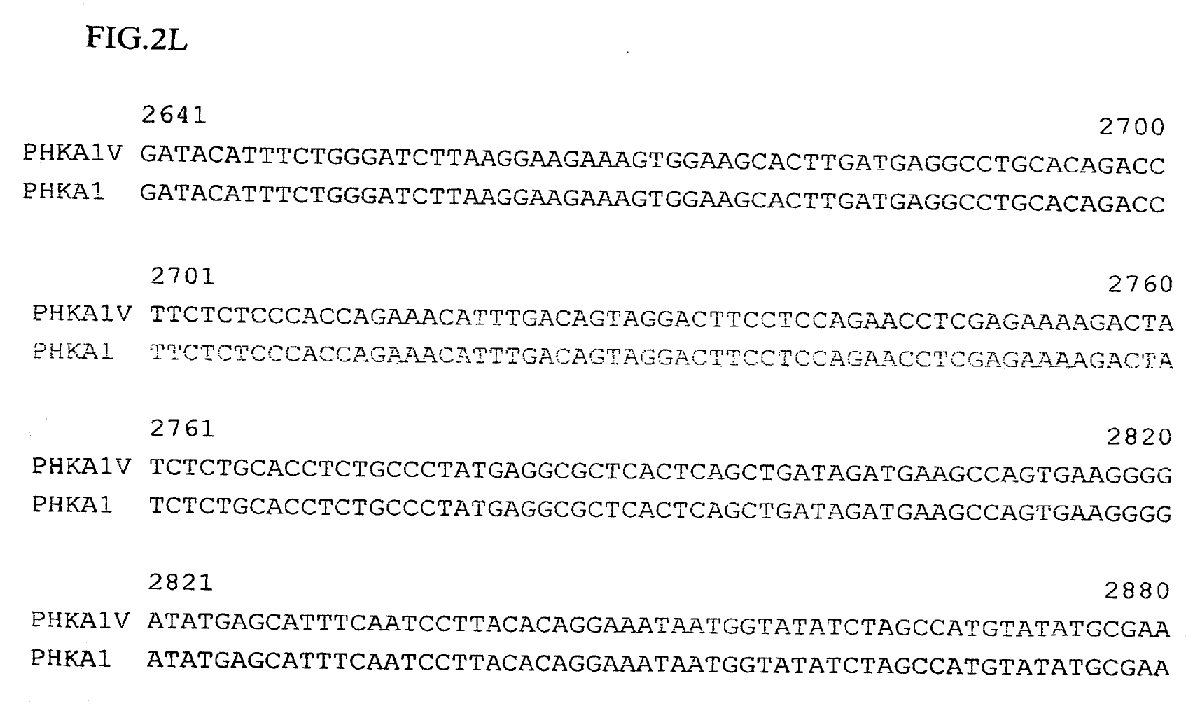

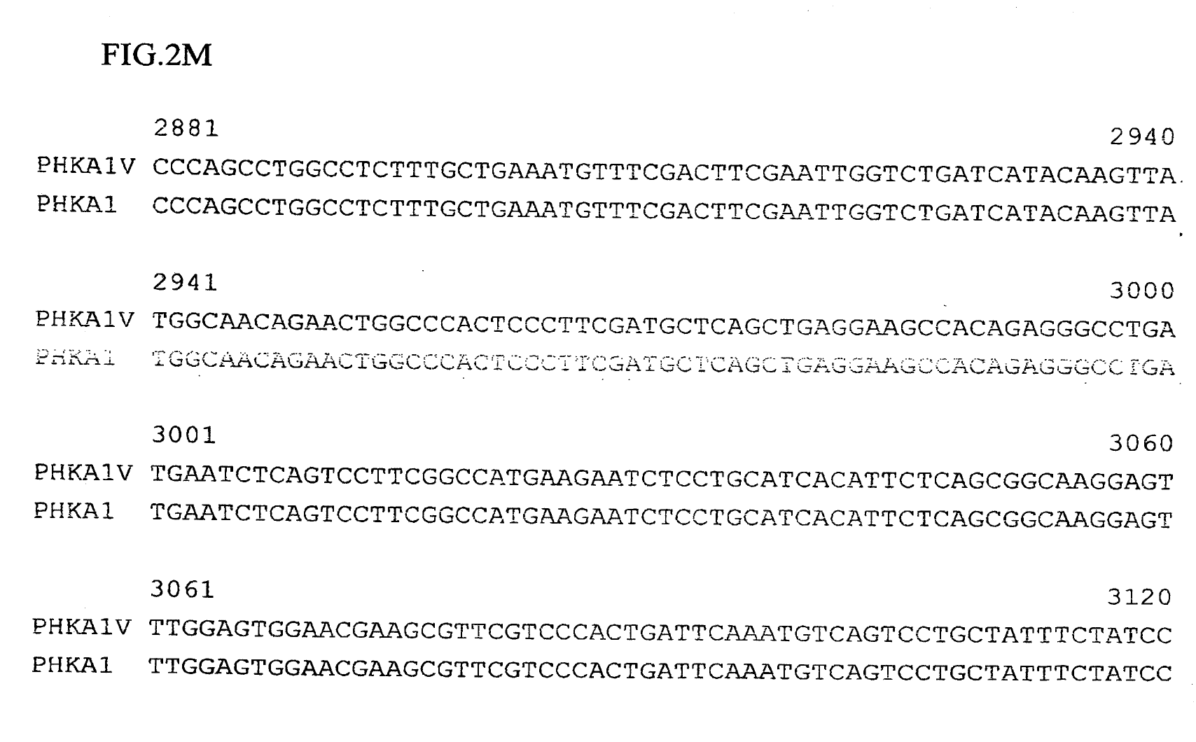

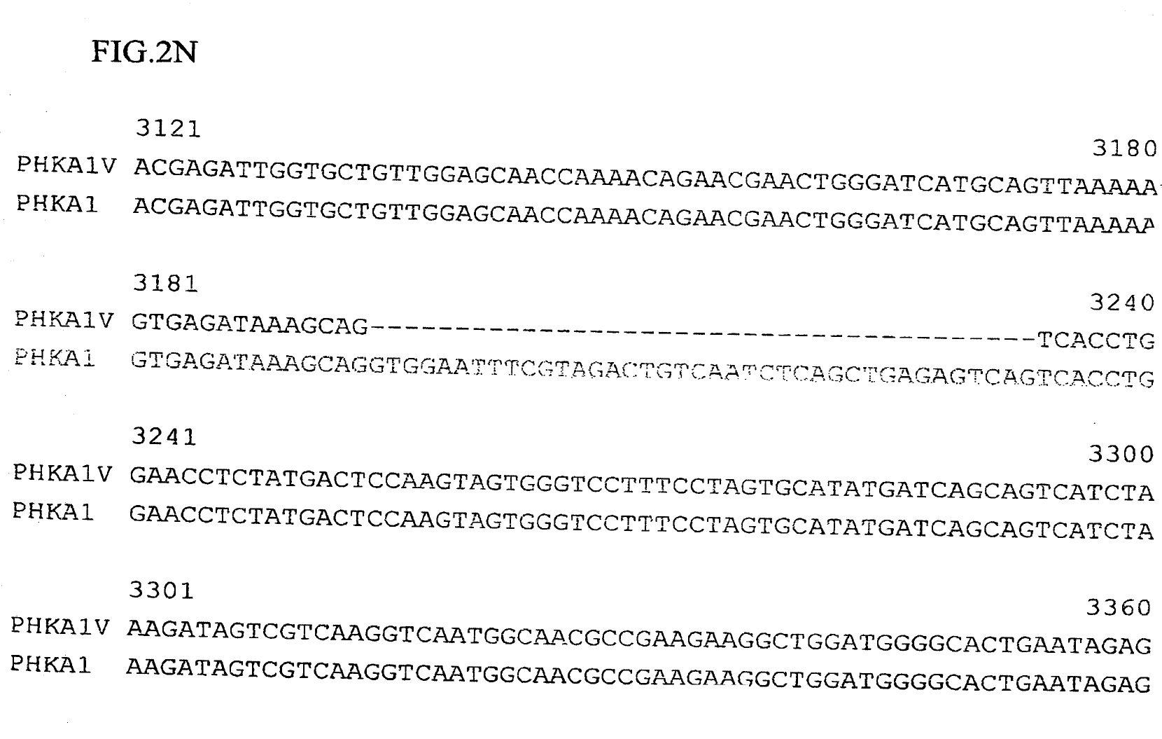

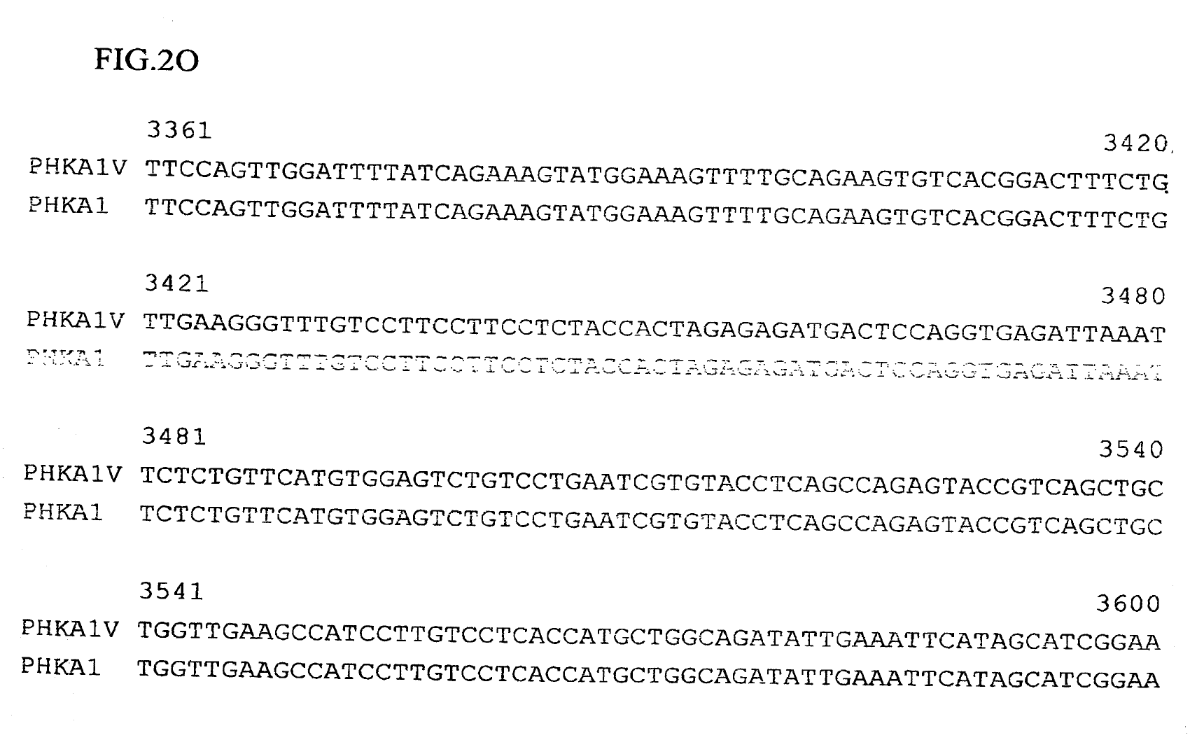

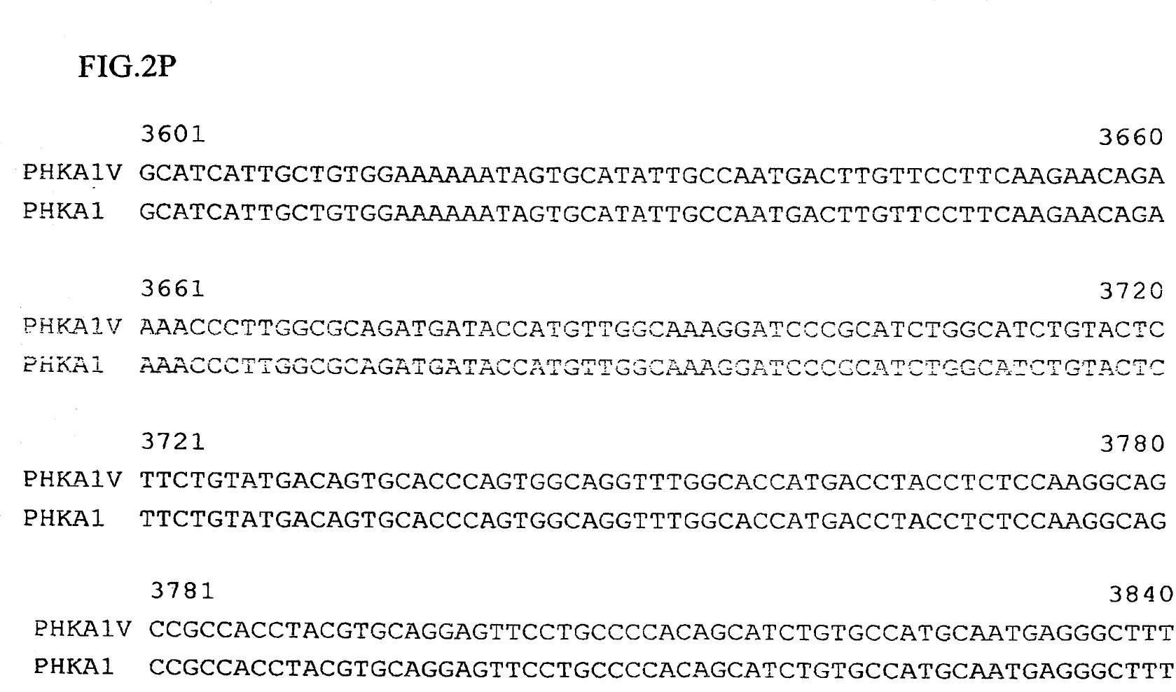

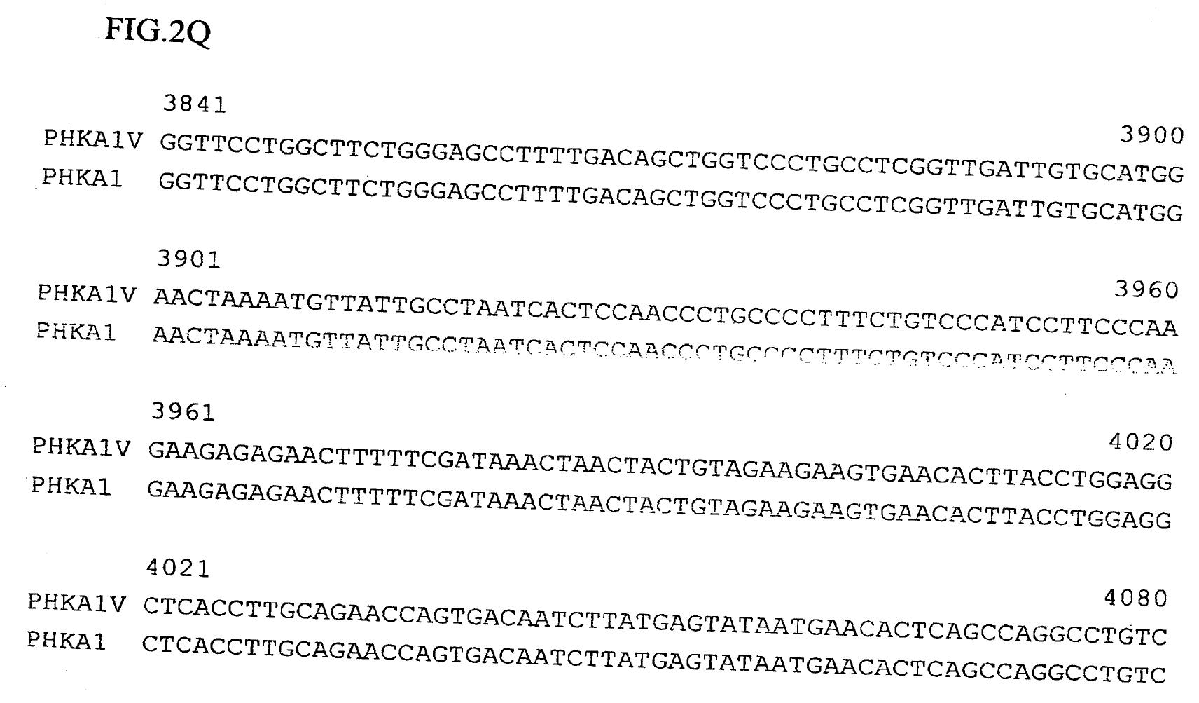

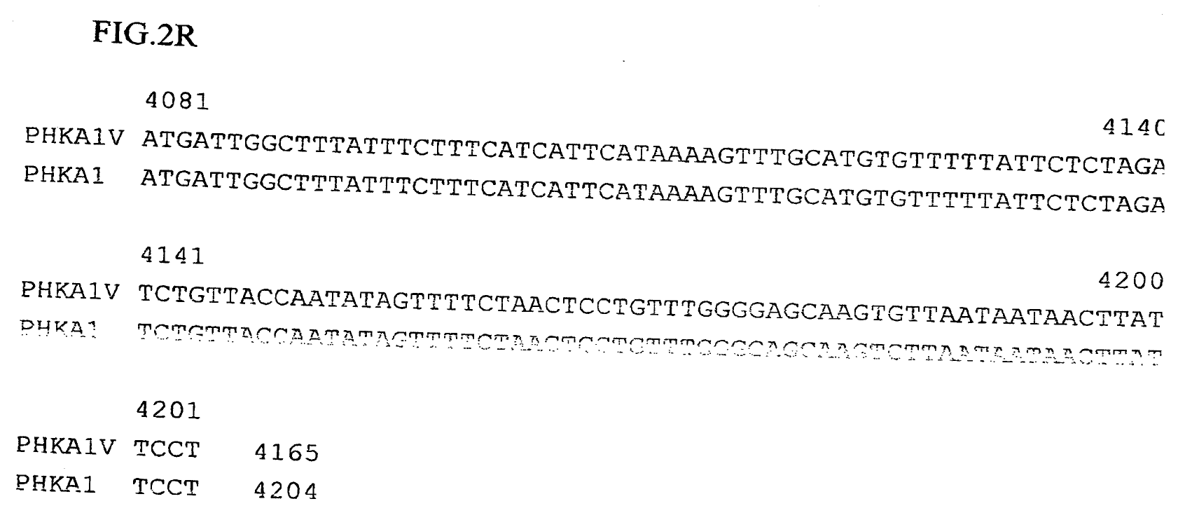

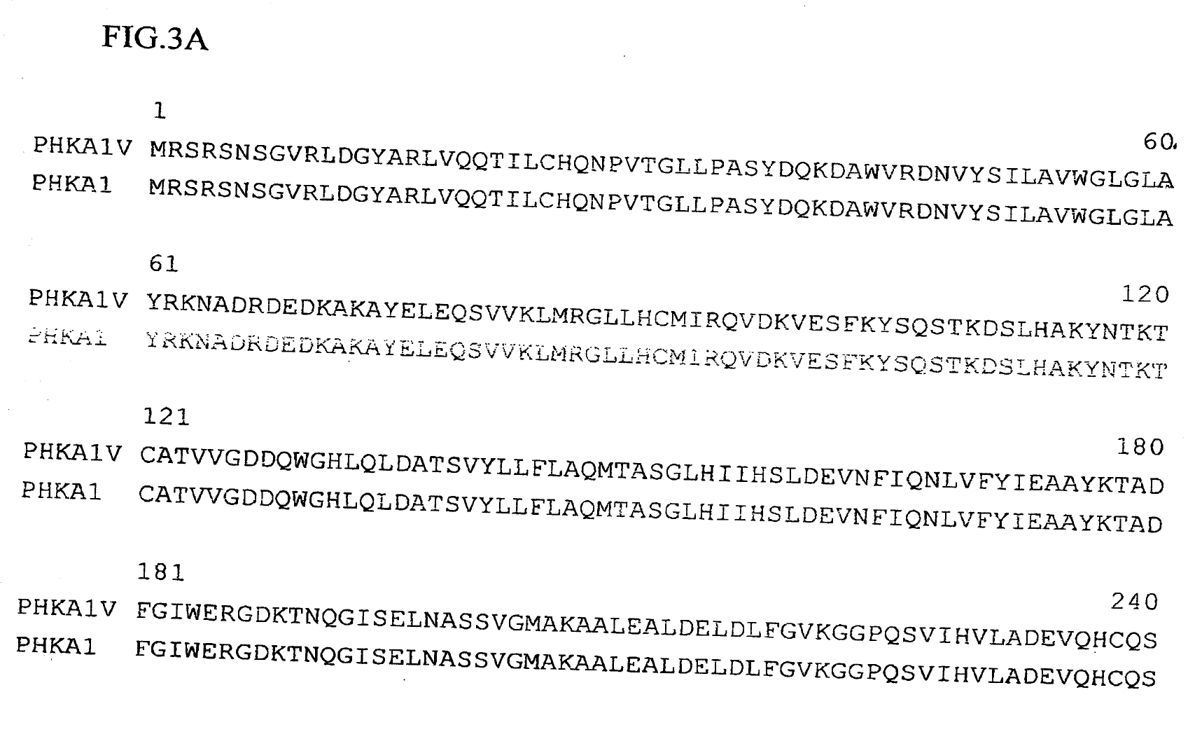

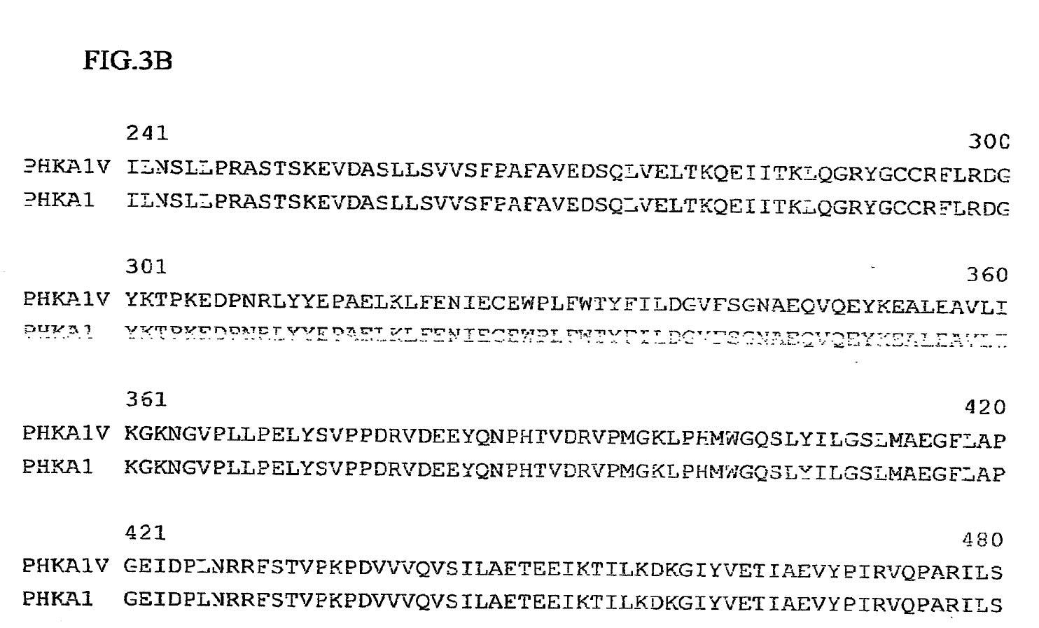

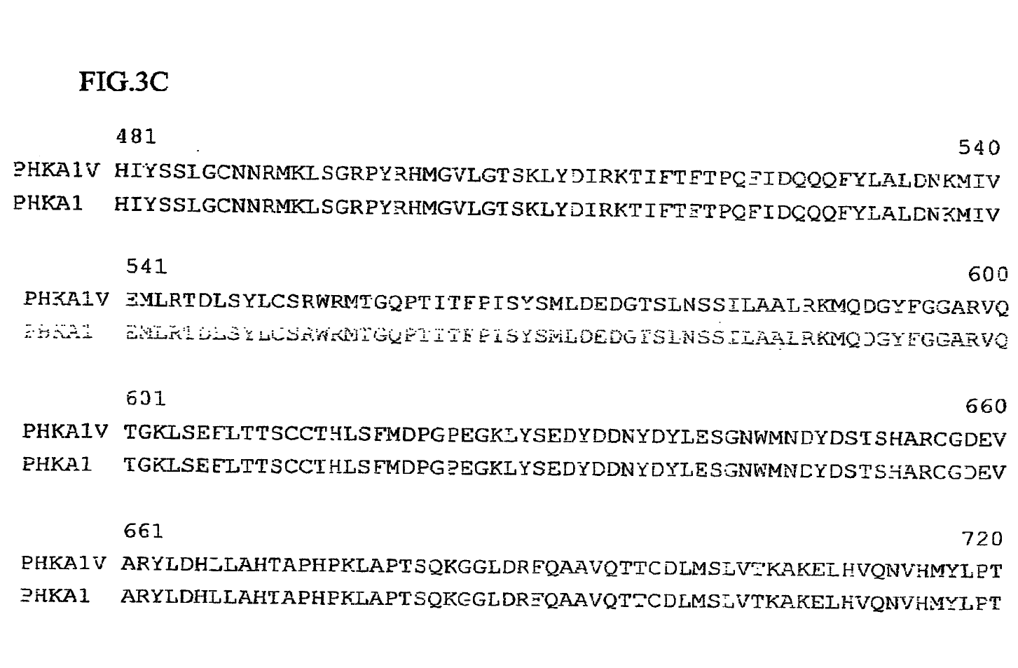

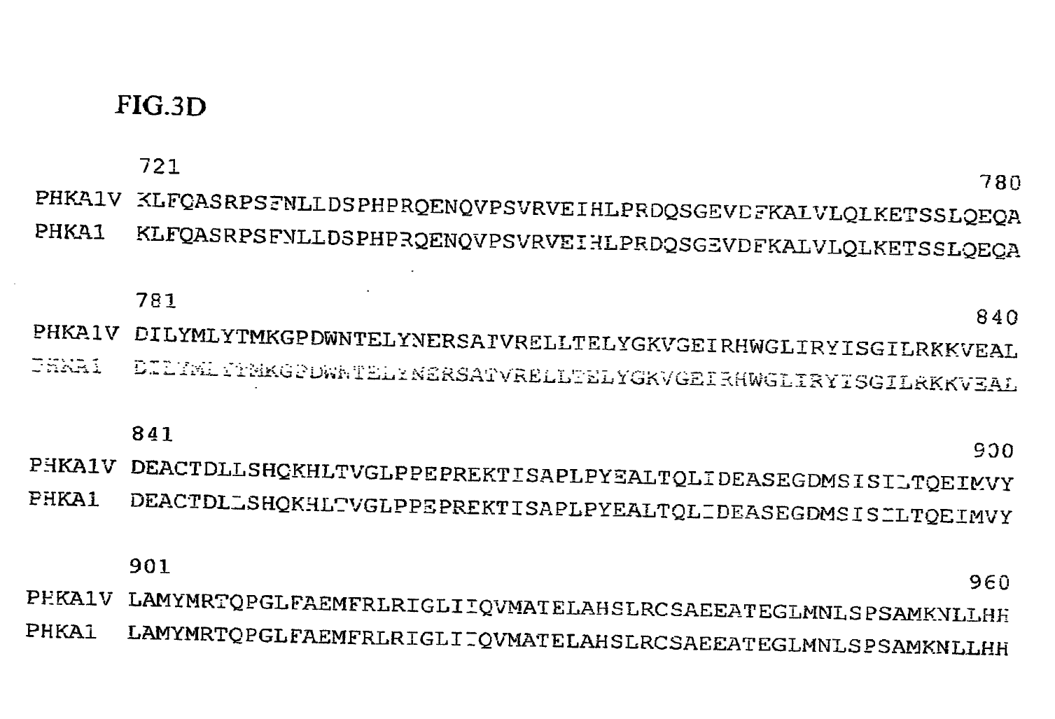

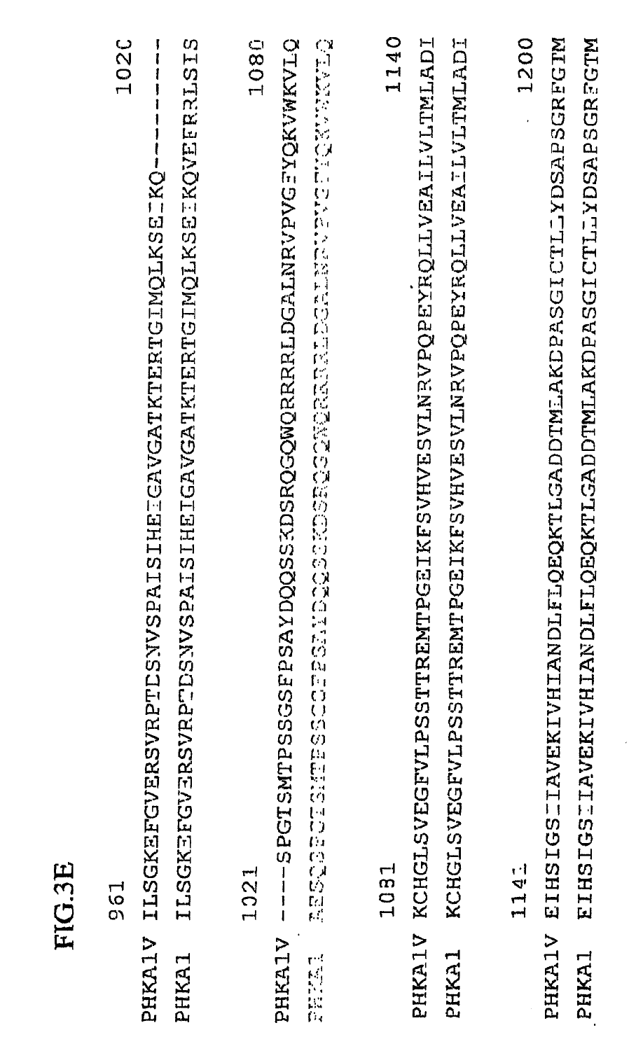

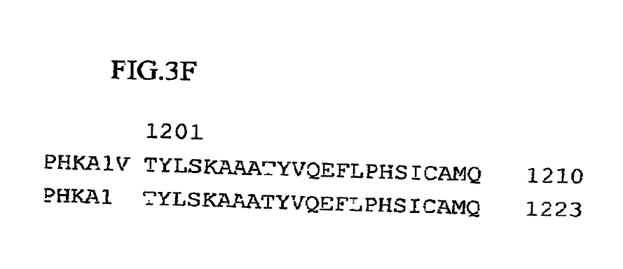

The full-length of the PHKA1V cDNA is a 4165 bp clone containing a 3630 bp open reading frame (ORF) extending from 162 bp to 3791 bp, which corresponds to an encoded protein of 1210 amino acid residues with a predicted molecular mass of 135.8 kDa. To determine the variation in sequence of PHKA1V cDNA clone, an alignment of PHKA1 nucleotide/amino acid sequence with PHKA1V was performed (FIGS. 2 and 3). One major genetic deletion was found in the aligned sequences, showing that PHKA1V is a 39 bp deletion in the sequence of PHKA1 from 3195 to 3233 bp. The lacking of 39 bp (corresponding to 13 amino acids) is an in-frame deletion in the amino acid sequence of PHKA1 and generates a polypeptide of 1210 amino acid residues of PHKA1V (FIG. 3).

In the present invention, a search of ESTs deposited in dbEST (Boguski et al. (1993) Nat Genet. 4: 332-3) at National Center of Biotechnology Information (NCBI) was performed to determine the distribution of PHKA1V in cancer tissues in silico. The result of in silico Northern analysis showed that ESTs such as EST (GenBank Accession Number BE619283) from lung large cell carcinoma, EST (GenBank Accession Number AA088294) from colon-tumor, EST (GenBank Accession Number BE909540) from pancreas epithelioid carcinoma, EST (GenBank Accession Number BG179601) from prostate adenocarcinoma, and EST (GenBank Accession Number BF928279) from nerve tumor were found to confirm the absence of 39 bp region on PHKA1V nucleotide sequence, suggesting that the absence of 39 bp nucleotide fragment located between 3194 to 3195 bp of PHKA1V may serve as a useful marker for diagnosing cancers. Therefore, any nucleotide fragments comprising 3194 to 3195 bp of PHKA1V may be used as probes for determining the presence of PHKA1V under highly stringent conditions. An alternative approach is that any set of primers for amplifying the fragment containing 3194 to 3195 bp of PHKA1V may be used for determining the presence of the variant.

According to the present invention, the polypeptide of the human PHKA1V and the fragments thereof may be produced via genetic engineering techniques. For instance, they may be produced by appropriate host cells which have been transformed by DNAs that code for the desired polypeptides or the fragments thereof. The nucleotide sequence encoding the polypeptide of the human PHKA1V or the fragments thereof is inserted into an appropriate expression vector, i.e., a vector which contains the necessary elements for the transcription and translation of the inserted coding sequence in a suitable host. The nucleic acid sequence is inserted into the vector in a manner such that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing sequences encoding the polypeptide of the human PHKA1V and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express the polypeptide-coding sequence. These include, but not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and most preferably, the bacterium is E. coli.

Alternatively, the polypeptide of the human PHKA1V or the fragments thereof may be synthesized by using chemical methods. For example, peptide synthesis can be performed using various solid-phase techniques (Roberge, J. Y. et al. (1995) Science 269: 202 to 204). Automated synthesis may be achieved by using the ABI 431A peptide synthesizer (Perkin-Elmer).

According to the present invention, the fragments of the polypeptide and nucleic acid sequences of PHKA1V are used as immunogens and primers or probes, respectively. Preferably, the purified fragments of the human PHKA1V are used. The fragments may be produced by enzymatic digestion, chemical cleavage of isolated or purified polypeptide or nucleic acid sequences, or chemical synthesis and then may be isolated or purified. Such isolated or purified fragments of the polypeptides and nucleic acid sequences can be used directed as immunogens and primers or probes, respectively.

The present invention further provides the antibodies which specifically bind one or more out-surface epitopes of the polypeptides of the human PHKA1V.

According to the present invention, immunization of mammals with immunogens described herein, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. Nos. 4,271,145 and 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci U S A 93: 12394-9; Liu et al., (1997) Nat Genet 16:328-9; Siffert et al., (1998) Nat Genet 18: 45 to 8; Lukas et al., (2001) Cancer Res 61: 3212 to 9). Although PHKA1V clone is isolated from lung cancers cDNA libraries, the in silico Northern analysis suggests that PHKA1V may serve as markers for the diagnosis of diseases associated with the deficiency of PHKA1 gene in a mammal, in particular, human cancers. Thus, the expression level of PHKA1V relative to PHKA1 may be a useful indicator for screening of patients suspected of having such diseases, and the index of relative expression level (mRNA or protein) may confer an increased susceptibility to the same.

Accordingly, the subject invention also provides methods for diagnosing diseases associated with the deficiency of PHKA1 gene in a mammal, in particular, cancers.

The method for diagnosing the diseases associated with the deficiency of PHKA1 gene may be performed by detecting the nucleotide sequence of the human PHKA1V of the invention which comprises the steps of: (1) extracting total RNA of cells obtained from a mammal; (2) amplifying the RNA by reverse transcriptase-polymerase chain reaction (RT-PCR) with a set of primers to obtain a cDNA comprising the fragments comprising nucleotides 3192 to 3197 of SEQ ID NO: 1; and (3) detecting whether the cDNA sample is obtained. If necessary, the amount of the obtained cDNA sample may be detected.

In the above embodiment, one of the primers may be designed to have a sequence comprising the nucleotides of SEQ ID NO: 1 containing nucleotides 3192 to 3197, and the other may be designed to have a sequence complementary to the nucleotides of SEQ ID NO: 1 at any other locations downstream of nucleotide 3197. Alternatively, one of the primers may be designed to have a sequence complementary to the nucleotides of SEQ ID NO: 1 containing nucleotides 3192 to 3197, and the other may be designed to have a sequence comprising the nucleotides of SEQ ID NO: 1 at any other locations upstream of nucleotide 3192. In this case, only PHKA1V will be amplified.

Alternatively, one of the primers may be designed to have a sequence comprising the nucleotides of SEQ ID NO: 1 upstream of nucleotide 3194 and the other may be designed to have a sequence complementary to the nucleotides of SEQ ID NO: 1 downstream of nucleotide 3195. Alternatively, one of the primers may be designed to have a sequence complementary to the nucleotides of SEQ ID NO: 1 upstream of nucleotide 3194 and the other may be designed to have a sequence comprising the nucleotides of SEQ ID NO: 1 downstream of nucleotide 3195. In this case, both PHKA1 and PHKA1V will be amplified. The length of the PCR fragment from PHKA1V will be 39 bp shorter than that from PHKA1.

Preferably, the primer of the invention contains 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected size of specific PCR fragments of PHKA1V can be used. PCR fragments are analyzed on a 1% agarose gel using five microliters (10%) of the amplified products. To determine the expression level of the gene variant, the intensity of the PCR products may be determined by using the Molecular Analyst program (version 1.4.1; Bio-Rad).

The RT-PCR experiment may be performed according to the manufacturer instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5X RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment for diagnosing the diseases associated with the deficiency of PHKA1 gene may be performed by detecting the nucleotide sequences of the human PHKA1V of the invention which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid of SEQ ID NO: 1 and the fragments thereof; and (4) detecting whether the cDNA sample hybridizes with the nucleic acid of SEQ ID NO: 1 or the fragments thereof. If necessary, the amount of hybridized sample may be detected.

The expression of gene variants can also be analyzed using Northern Blot hybridization approach. Specific fragments comprising nucleotides 3192 to 3197 of the PHKA1V may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency associated with PHKA1 gene may also be performed by detecting the polypeptide encoded by the human PHKA1V of the invention. For instance, the polypeptide in protein samples obtained from the mammal suspected of having such diseases may be determined by, but not limited to, the immunoassay wherein the antibody specifically binding to the polypeptide of the invention is brought into contact with the protein samples, and the antibody-polypeptide complex is detected. If necessary, the amount of antibody-polypeptide complex can be determined.

The polypeptide of the PHKA1V may be expressed in prokaryotic cells by using suitable prokaryotic expression vectors. The cDNA fragments of PHKA1V gene encoding the amino acid coding sequence may be PCR amplified using primer set with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 h. The bacterially-expressed proteins may be purified.

The polypeptide of the gene variant may also be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% Co₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectamine Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins (PHKA1V) may be generated using hybridoma technique according to the conventional methods (de StGroth and Scheidegger, (1980) J Immunol Methods 35:1-21; Cote et al. (1983) Proc Natl Acad Sci U S A 80: 2026-30; and Kozbor et al. (1985) J Immunol Methods 81:31-42).

According to the present invention, the presence of the polypeptide of the gene variant in samples obtained from the mammal suspected of having diseases associated with the deficiency of PHKA1 gene may be determined by, but not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 h at 100 V (e.g., Bio-Rad). The proteins can be immunoblotted with specific antibodies. For example, membrane blotted with extracted proteins may be blocked with suitable buffers such as 3% solution of B SA or 3% solution of nonfat milk powder in TB ST buffer (10 mM Tris-HCl, pH 8.0, 150 mM NaCl, 0.1% Tween 20) and incubated with monoclonal antibody directed against the polypeptides of gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human lung (normal, SCLC, squamous cell lung cancer and large cell lung cancer) cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative PHKA1V gene were identified during the course of EST generation.

### Isolation of cDNA Clones

Three identical cDNA clone exhibiting EST sequences similar to the PHKA1 gene were isolated from lung cancers cDNA libraries and named PHKA1V. The inserts of these clones were subsequently excised in vivo from the λZAP Express vector using the ExAssist/XLOLR helper phage system (Stratagene). Phagemid particles were excised by coinfecting XL 1-BLUE MRF' cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution (Northern) Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### REFERENCES

Altschul et al., Gapped BLAST and PSI-BLAST: a new generation of protein database search programs, Nucleic Acids Res, 25: 3389-3402, (1997).

Ausubel et al., Current protocols in Molecular Biology, John Wiley & Sons, New York N.Y., ch. 9, 13, and 16, (1995).

Boguski et al., dbEST—database for “expressed sequence tags”. Nat Genet. 4: 332-3, (1993).

Brushia and Walsh, Phosphorylase kinase: the complexity of its regulation is reflected in the complexity of its structure. Front Biosci, 4:D618-41, (1999).

Carney, The biology of lung cancer. Curr. Opin. Oncol. 4: 292-8, (1992a).

Carney, Biology of small-cell lung cancer. Lancet 339: 843-6, (1992b).

Cote et al., Generation of human monoclonal antibodies reactive with cellular antigens, Proc Natl Acad Sci U S A 80: 2026-30 (1983).

de StGroth and Scheidegger, Production of monoclonal antibodies: strategy and tactics, J Immunol Methods 35:1-21, (1980).

Francke et al., Assignment of human genes for phosphorylase kinase subunits alpha (PHKA) to Xq12-q13 and beta (PHKB) to 16q12-q13. Am J Hum Genet, 45:276-82, (1989).

Gish and States, Identification of protein coding regions by database similarity search, Nat Genet, 3:266-272, (1993).

Ihde and Minna, Non-small cell lung cancer. Part II: Treatment. Curr. Probl. Cancer 15: 105-54, (1991).

Kozbor et al., Specific immunoglobulin production and enhanced tumorigenicity following ascites growth of human hybridomas, J Immunol Methods, 81:31-42 (1985).

Levin et al. Identification of novel regions of altered DNA copy number in small cell lung tumors. Genes Chromosomes Cancer, 13:175-85, (1995).

Liu et al., Silent mutation induces exon skipping of fibrillin-1 gene in Marfan syndrome. Nat Genet 16:328-9, (1997).

Lukas et al., Alternative and aberrant messenger RNA splicing of the mdm2 oncogene in invasive breast cancer. Cancer Res 61:3212-9, (2001).

Roberge et al., A strategy for a convergent synthesis of N-linked glycopeptides on a solid support. Science 269:202-4, (1995).

Sambrook, J. Cold Spring Harbor Press, Plainview N.Y., ch. 4, 8, and 16-17.

Sethi, Science, medicine, and the future. Lung cancer, BMJ, 314: 652-655, (1997)

Siffert et al., Association of a human G-protein beta3 subunit variant with hypertension. Nat Genet, 18:45-8, (1998).

Simpson, A. J. G. GenBank accession # BF928279

Smyth et al., The impact of chemotherapy on small cell carcinoma of the bronchus. Q J Med, 61: 969-76, (1986).

Stallings-Mann et al., Alternative splicing of exon 3 of the human growth hormone receptor is the result of an unusual genetic polymorphism. Proc Natl Acad Sci U S A 93:12394-9, (1996).

Strausberg, R. GenBank accession # BE619283, BE909540, BG179601

Sundareshan and Augustus M, Cytogenetics of non-small cell lung cancer: simple technique for obtaining high quality chromosomes by fine needle aspirate cultures. Cancer Genet Cytogenet 91:53-60, (1996).

Wilson, R. K. GenBank accession # AA088294

Yano et al., Evaluation of glycogen level in human lung carcinoma tissues by an infrared spectroscopic method. Cancer Lett, 110:29-34, (1996).

SEQ ID NO: 1

| | |
|---|---|
| GCCGCCGGGCGCCAGGCCTGAGCGGTGGGAGGGCTCTGCGGGGCCTGGTGTTCAGGCGTC | 60 |
| CCACCACGAGGGTGGAGCAGCGTTGGATACTTGTTCCTTAGGGACCGAAGCTCCGGTGGC | 120 |
| ACCCGGGCTATTTCTCAGAGGACAATTAGTAACGTGTCGCCATGAGGAGCCGGAGTAACT | 180 |
| CCGGGGTCCGGCTGGACGGCTACGCTCGACTGGTGCAACAGACCATCCTGTGCCATCAGA | 240 |
| ATCCAGTGACTGGCTTGCTTCCAGCCAGCTATGATCAGAAAGATGCTTGGGTCCGAGATA | 300 |
| ATGTGTACAGCATCTTGGCTGTGTGGGGTTTGGGCCTGGCCTATCGGAAGAATGCAGACC | 360 |
| GGGATGAGGATAAGGCAAAGGCCTATGAATTGGAGCAGAGTGTAGTGAAGCTGATGAGAG | 420 |
| GACTACTGCACTGCATGATCAGACAGGTGGATAAAGTAGAATCCTTCAAATATAGTCAGA | 480 |
| GTACTAAGGATAGCCTCCATGCAAAGTACAACACCAAAACCTGTGCCACTGTAGTGGGTG | 540 |
| ATGATCAATGGGGACACCTGCAGTTGGATGCTACCTCTGTGTACCTGCTCTTCTTAGCCC | 600 |
| AAATGACTGCCTCAGGACTCCATATCATCCACAGCCTAGATGAAGTCAATTTCATACAGA | 660 |
| ACCTTGTGTTTTACATTGAAGCTGCATATAAAACTGCTGACTTCGGGATATGGGAACGTG | 720 |
| GAGACAAGACCAACCAAGGGATCTCAGAGTTGAATGCCAGTTCAGTTGGAATGGCAAAGG | 780 |
| CAGCCCTGGAAGCATTAGATGAACTGGATCTGTTTGGTGTGAAAGGTGGGCCTCAATCAG | 840 |
| TTATCCATGTCCTGGCTGATGAAGTACAGCACTGCCAGTCTATCCTAAATTCACTACTGC | 900 |
| CCCGTGCTTCAACATCAAAAGAGGTTGATGCTAGTCTACTCTCAGTGGTTTCCTTCCCTG | 960 |
| CCTTTGCAGTAGAGGATAGCCAGTTGGTGGAGCTCACAAAACAGGAAATCATCACCAAGC | 1020 |
| TTCAGGGTCGTTATGGTTGCTGTCGCTTTCTACGAGATGGATATAAAACTCCTAAAGAGG | 1080 |
| ATCCCAATCGTCTGTACTATGAACCAGCTGAGCTGAAGCTATTTGAAAACATTGAGTGTG | 1140 |
| AATGGCCATTGTTCTGGACATACTTTATTCTTGATGGGGTCTTCAGTGGCAATGCAGAAC | 1200 |
| AGGTTCAAGAATATAAAGAGGCTCTTGAAGCAGTCCTCATCAAGGGCAAAAATGGAGTCC | 1260 |
| CACTTCTGCCAGAGCTGTACAGTGTTCCTCCTGACAGGGTCGATGAAGAATATCAGAATC | 1320 |
| CTCACACTGTGGACCGAGTCCCCATGGGGAAATTGCCTCACATGTGGGGTCAGTCTCTAT | 1380 |
| ACATTTTAGGAAGCTTGATGGCAGAGGGATTTTTAGCCCCTGGAGAAATTGATCCCCTGA | 1440 |
| ATCGCAGGTTTTCTACTGTACCGAAGCCCGATGTTGTGGTTCAAGTCTCCATTCTAGCTG | 1500 |
| AAACAGAAGAAATCAAGACCATTTTGAAGGACAAGGGAATTTACGTGGAGACCATTGCTG | 1560 |
| AGGTATACCCCATCAGAGTACAACCAGCTCGTATTCTCAGCCACATTTATTCCAGCCTAG | 1620 |
| GATGCAACAATAGAATGAAACTCAGTGGACGACCCTACAGACACATGGGAGTGCTTGGAA | 1680 |
| CTTCAAAACTCTATGACATTCGGAAAACTATCTTTACTTTCACTCCACAGTTTATAGACC | 1740 |
| AGCAACAGTTCTACCTGGCTCTGGACAACAAGATGATAGTGGAAATGCTTAGAACAGACC | 1800 |
| TCTCCTACCTCTGTAGCCGCTGGCGGATGACAGGCCAGCCCACCATCACCTTCCCCATCT | 1860 |
| CATACAGCATGCTTGATGAAGATGGAACAAGCTTGAATTCAAGTATCCTGGCAGCACTCC | 1920 |
| GAAAAATGCAAGATGGGTATTTTGGTGGGGCAAGGGTTCAAACAGGTAAATTGTCAGAGT | 1980 |
| TTTTGACAACATCTTGTTGCACACACTTGAGCTTCATGGACCCTGGACCTGAGGGTAAGC | 2040 |
| TGTACAGTGAAGATTATGATGACAACTATGATTACCTGGAATCTGGCAACTGGATGAATG | 2100 |
| ATTATGATTCAACCAGTCATGCTCGCTGTGGTGATGAAGTTGCTCGTTATTTAGATCACC | 2160 |
| TTTTGGCGCACACTGCTCCCCATCCTAAACTAGCCCCTACCTCACAGAAGGGAGGGCTAG | 2220 |
| ATCGGTTCCAAGCTGCTGTGCAAACAACCTGCGACTTAATGTCCTTGGTGACCAAGGCCA | 2280 |
| AGGAACTGCATGTACAGAATGTTCACATGTATCTTCCTACGAAGTTATTTCAGGCTTCCC | 2340 |
| GGCCTTCATTCAACTTACTTGATTCACCTCATCCCCGACAGGAGAACCAGGTTCCCTCTG | 2400 |
| TTCGTGTAGAAATACATCTTCCTAGAGACCAGTCTGGGGAGGTGGACTTTAAAGCACTGG | 2460 |
| TTTTACAGTTGAAGGAGACCTCAAGCTTACAGGAACAAGCTGATATCCTCTATATGCTGT | 2520 |
| ATACTATGAAAGGACCTGACTGGAACACTGAATTGTATAATGAACGGAGTGCTACAGTGA | 2580 |
| GAGAGCTTCTTACCGAGCTGTATGGCAAAGTGGGAGAAATTCGTCACTGGGGCCTGATCC | 2640 |
| GATACATTTCTGGGATCTTAAGGAAGAAAGTGGAAGCACTTGATGAGGCCTGCACAGACC | 2700 |
| TTCTCTCCCACCAGAAACATTTGACAGTAGGACTTCCTCCAGAACCTCGAGAAAAGACTA | 2760 |
| TCTCTGCACCTCTGCCCTATGAGGCGCTCACTCAGCTGATAGATGAAGCCAGTGAAGGGG | 2820 |
| ATATGAGCATTTCAATCCTTACACAGGAAATAATGGTATATCTAGCCATGTATATGCGAA | 2880 |
| CCCAGCCTGGCCTCTTTGCTGAAATGTTTCGACTTCGAATTGGTCTGATCATACAAGTTA | 2940 |
| TGGCAACAGAACTGGCCCACTCCCTTCGATGCTCAGCTGAGGAAGCCACAGAGGGCCTGA | 3000 |
| TGAATCTCAGTCCTTCGGCCATGAAGAATCTCCTGCATCACATTCTCAGCGGCAAGGAGT | 3060 |
| TTGGAGTGGAACGAAGCGTTCGTCCCACTGATTCAAATGTCAGTCCTGCTATTTCTATCC | 3120 |
| ACGAGATTGGTGCTGTTGGAGCAACCAAAACAGAACGAACTGGGATCATGCAGTTAAAAA | 3180 |
| GTGAGATAAAGCAGTCACCTGGAACCTCTATGACTCCAAGTAGTGGGTCCTTTCCTAGTG | 3240 |
| CATATGATCAGCAGTCATCTAAAGATAGTCGTCAAGGTCAATGGCAACGCCGAAGAAGGC | 3300 |
| TGGATGGGGCACTGAATAGAGTTCCAGTTGGATTTTATCAGAAAGTATGGAAAGTTTTGC | 3360 |
| AGAAGTGTCACGGACTTTCTGTTGAAGGGTTTGTCCTTCCTTCCTCTACCACTAGAGAGA | 3420 |
| TGACTCCAGGTGAGATTAAATTCTCTGTTCATGTGGAGTCTGTCCTGAATCGTGTACCTC | 3480 |
| AGCCAGAGTACCGTCAGCTGCTGGTTGAAGCCATCCTTGTCCTCACCATGCTGGCAGATA | 3540 |
| TTGAAATTCATAGCATCGGAAGCATCATTGCTGTGGAAAAAATAGTGCATATTGCCAATG | 3600 |
| ACTTGTTCCTTCAAGAACAGAAAACCCTTGGCGCAGATGATACCATGTTGGCAAAGGATC | 3660 |
| CCGCATCTGGCATCTGTACTCTTCTGTATGACAGTGCACCCAGTGGCAGGTTTGGCACCA | 3720 |
| TGACCTACCTCTCCAAGGCAGCCGCCACCTACGTGCAGGAGTTCCTGCCCCACAGCATCT | 3780 |
| GTGCCATGCAATGAGGGCTTTGGTTCCTGGCTTCTGGGAGCCTTTTGACAGCTGGTCCCT | 3840 |
| GCCTCGGTTGATTGTGCATGGAACTAAAATGTTATTGCCTAATCACTCCAACCCTGCCCC | 3900 |
| TTTCTGTCCCATCCTTCCCAAGAAGAGAGAACTTTTTCGATAAACTAACTACTGTAGAAG | 3960 |
| AAGTGAACACTTACCTGGAGGCTCACCTTGCAGAACCAGTGACAATCTTATGAGTATAAT | 4020 |
| GAACACTCAGCCAGGCCTGTCATGATTGGCTTTATTTCTTTCATCATTCATAAAAGTTTG | 4080 |
| CATGTGTTTTTATTCTCTAGATCTGTTACCAATATAGTTTTCTAACTCCTGTTTGGGGAG | 4140 |
| CAAGTGTTAATAATAACTTATTCCT 4165 | |

| SEQ ID NO: 2 | |
|---|---|
| Met Arg Ser Arg Ser Asn Ser Gly Val Arg | 10 |
| Leu Asp Gly Tyr Ala Arg Leu Val Gln Gln | 20 |
| Thr Ile Leu Cys His Gln Asn Pro Val Thr | 30 |
| Gly Leu Leu Pro Ala Ser Tyr Asp Gln Lys | 40 |
| Asp Ala Trp Val Arg Asp Asn Val Tyr Ser | 50 |
| Ile Leu Ala Val Trp Gly Leu Gly Leu Ala | 60 |
| Tyr Arg Lys Asn Ala Asp Arg Asp Glu Asp | 70 |
| Lys Ala Lys Ala Tyr Glu Leu Glu Gln Ser | 80 |
| Val Val Lys Leu Met Arg Gly Leu Leu His | 90 |
| Cys Met Ile Arg Gln Val Asp Lys Val Glu | 100 |
| Ser Phe Lys Tyr Ser Gln Ser Thr Lys Asp | 110 |
| Ser Leu His Ala Lys Tyr Asn Thr Lys Thr | 120 |
| Cys Ala Thr Val Val Gly Asp Asp Gln Trp | 130 |
| Gly His Leu Gln Leu Asp Ala Thr Ser Val | 140 |
| Tyr Leu Leu Phe Leu Ala Gln Met Thr Ala | 150 |
| Ser Gly Leu His Ile Ile His Ser Leu Asp | 160 |
| Glu Val Asn Phe Ile Gln Asn Leu Val Phe | 170 |
| Tyr Ile Glu Ala Ala Tyr Lys Thr Ala Asp | 180 |
| Phe Gly Ile Trp Glu Arg Gly Asp Lys Thr | 190 |
| Asn Gln Gly Ile Ser Glu Leu Asn Ala Ser | 200 |
| Ser Val Gly Met Ala Lys Ala Ala Leu Glu | 210 |
| Ala Leu Asp Glu Leu Asp Leu Phe Gly Val | 220 |
| Lys Gly Gly Pro Gln Ser Val Ile His Val | 230 |
| Leu Ala Asp Glu Val Gln His Cys Gln Ser | 240 |
| Ile Leu Asn Ser Leu Leu Pro Arg Ala Ser | 250 |
| Thr Ser Lys Glu Val Asp Ala Ser Leu Leu | 260 |
| Ser Val Val Ser Phe Pro Ala Phe Ala Val | 270 |
| Glu Asp Ser Gln Leu Val Glu Leu Thr Lys | 280 |
| Gln Glu Ile Ile Thr Lys Leu Gln Gly Arg | 290 |
| Tyr Gly Cys Cys Arg Phe Leu Arg Asp Gly | 300 |
| Tyr Lys Thr Pro Lys Glu Asp Pro Asn Arg | 310 |
| Leu Tyr Tyr Glu Pro Ala Glu Leu Lys Leu | 320 |
| Phe Glu Asn Ile Glu Cys Glu Trp Pro Leu | 330 |
| Phe Trp Thr Tyr Phe Ile Leu Asp Gly Val | 340 |
| Phe Ser Gly Asn Ala Glu Gln Val Gln Glu | 350 |
| Tyr Lys Glu Ala Leu Glu Ala Val Leu Ile | 360 |
| Lys Gly Lys Asn Gly Val Pro Leu Leu Pro | 370 |
| Glu Leu Tyr Ser Val Pro Pro Asp Arg Val | 380 |
| Asp Glu Glu Tyr Gln Asn Pro His Thr Val | 390 |
| Asp Arg Val Pro Met Gly Lys Leu Pro His | 400 |
| Met Trp Gly Gln Ser Leu Tyr Ile Leu Gly | 410 |
| Ser Leu Met Ala Glu Gly Phe Leu Ala Pro | 420 |
| Gly Glu Ile Asp Pro Leu Asn Arg Arg Phe | 430 |
| Ser Thr Val Pro Lys Pro Asp Val Val Val | 440 |
| Gln Val Ser Ile Leu Ala Glu Thr Glu Glu | 450 |
| Ile Lys Thr Ile Leu Lys Asp Lys Gly Ile | 460 |
| Tyr Val Glu Thr Ile Ala Glu Val Tyr Pro | 470 |
| Ile Arg Val Gln Pro Ala Arg Ile Leu Ser | 480 |
| His Ile Tyr Ser Ser Leu Gly Cys Asn Asn | 490 |
| Arg Met Lys Leu Ser Gly Arg Pro Tyr Arg | 500 |
| His Met Gly Val Leu Gly Thr Ser Lys Leu | 510 |
| Tyr Asp Ile Arg Lys Thr Ile Phe Thr Phe | 520 |
| Thr Pro Gln Phe Ile Asp Gln Gln Gln Phe | 530 |
| Tyr Leu Ala Leu Asp Asn Lys Met Ile Val | 540 |
| Glu Met Leu Arg Thr Asp Leu Ser Tyr Leu | 550 |
| Cys Ser Arg Trp Arg Met Thr Gly Gln Pro | 560 |
| Thr Ile Thr Phe Pro Ile Ser Tyr Ser Met | 570 |
| Leu Asp Glu Asp Gly Thr Ser Leu Asn Ser | 580 |
| Ser Ile Leu Ala Ala Leu Arg Lys Met Gln | 590 |
| Asp Gly Tyr Phe Gly Gly Ala Arg Val Gln | 600 |
| Thr Gly Lys Leu Ser Glu Phe Leu Thr Thr | 610 |
| Ser Cys Cys Thr His Leu Ser Phe Met Asp | 620 |
| Pro Gly Pro Glu Gly Lys Leu Tyr Ser Glu | 630 |
| Asp Tyr Asp Asp Asn Tyr Asp Tyr Leu Glu | 640 |
| Ser Gly Asn Trp Met Asn Asp Tyr Asp Ser | 650 |
| Thr Ser His Ala Arg Cys Gly Asp Glu Val | 660 |
| Ala Arg Tyr Leu Asp His Leu Leu Ala His | 670 |
| Thr Ala Pro His Pro Lys Leu Ala Pro Thr | 680 |
| Ser Gln Lys Gly Gly Leu Asp Arg Phe Gln | 690 |
| Ala Ala Val Gln Thr Thr Cys Asp Leu Met | 700 |
| Ser Leu Val Thr Lys Ala Lys Glu Leu His | 710 |
| Val Gln Asn Val His Met Tyr Leu Pro Thr | 720 |
| Lys Leu Phe Gln Ala Ser Arg Pro Ser Phe | 730 |
| Asn Leu Leu Asp Ser Pro His Pro Arg Gln | 740 |
| Glu Asn Gln Val Pro Ser Val Arg Val Glu | 750 |
| Ile His Leu Pro Arg Asp Gln Ser Gly Glu | 760 |
| Val Asp Phe Lys Ala Leu Val Leu Gln Leu | 770 |
| Lys Glu Thr Ser Ser Leu Gln Glu Gln Ala | 780 |
| Asp Ile Leu Tyr Met Leu Tyr Thr Met Lys | 790 |
| Gly Pro Asp Trp Asn Thr Glu Leu Tyr Asn | 800 |
| Glu Arg Ser Ala Thr Val Arg Glu Leu Leu | 810 |
| Thr Glu Leu Tyr Gly Lys Val Gly Glu Ile | 820 |
| Arg His Trp Gly Leu Ile Arg Tyr Ile Ser | 830 |
| Gly Ile Leu Arg Lys Lys Val Glu Ala Leu | 840 |
| Asp Glu Ala Cys Thr Asp Leu Leu Ser His | 850 |
| Gln Lys His Leu Thr Val Gly Leu Pro Pro | 860 |
| Glu Pro Arg Glu Lys Thr Ile Ser Ala Pro | 870 |
| Leu Pro Tyr Glu Ala Leu Thr Gln Leu Ile | 880 |
| Asp Glu Ala Ser Glu Gly Asp Met Ser Ile | 890 |
| Ser Ile Leu Thr Gln Glu Ile Met Val Tyr | 900 |
| Leu Ala Met Tyr Met Arg Thr Gln Pro Gly | 910 |
| Leu Phe Ala Glu Met Phe Arg Leu Arg Ile | 920 |
| Gly Leu Ile Ile Gln Val Met Ala Thr Glu | 930 |
| Leu Ala His Ser Leu Arg Cys Ser Ala Glu | 940 |
| Glu Ala Thr Glu Gly Leu Met Asn Leu Ser | 950 |
| Pro Ser Ala Met Lys Asn Leu Leu His His | 960 |
| Ile Leu Ser Gly Lys Glu Phe Gly Val Glu | 970 |
| Arg Ser Val Arg Pro Thr Asp Ser Asn Val | 980 |
| Ser Pro Ala Ile Ser Ile His Glu Ile Gly | 990 |
| Ala Val Gly Ala Thr Lys Thr Glu Arg Thr | 1000 |
| Gly Ile Met Gln Leu Lys Ser Glu Ile Lys | 1010 |
| Gln Ser Pro Gly Thr Ser Met Thr Pro Ser | 1020 |
| Ser Gly Ser Phe Pro Ser Ala Tyr Asp Gln | 1030 |
| Gln Ser Ser Lys Asp Ser Arg Gln Gly Gln | 1040 |
| Trp Gln Arg Arg Arg Arg Leu Asp Gly Ala | 1050 |
| Leu Asn Arg Val Pro Val Gly Phe Tyr Gln | 1060 |
| Lys Val Trp Lys Val Leu Gln Lys Cys His | 1070 |
| Gly Leu Ser Val Glu Gly Phe Val Leu Pro | 1080 |
| Ser Ser Thr Thr Arg Glu Met Thr Pro Gly | 1090 |
| Glu Ile Lys Phe Ser Val His Val Glu Ser | 1100 |
| Val Leu Asn Arg Val Pro Gln Pro Glu Tyr | 1110 |
| Arg Gln Leu Leu Val Glu Ala Ile Leu Val | 1120 |
| Leu Thr Met Leu Ala Asp Ile Glu Ile His | 1130 |
| Ser Ile Gly Ser Ile Ile Ala Val Glu Lys | 1140 |
| Ile Val His Ile Ala Asn Asp Leu Phe Leu | 1150 |
| Gln Glu Gln Lys Thr Leu Gly Ala Asp Asp | 1160 |
| Thr Met Leu Ala Lys Asp Pro Ala Ser Gly | 1170 |
| Ile Cys Thr Leu Leu Tyr Asp Ser Ala Pro | 1180 |
| Ser Gly Arg Phe Gly Thr Met Thr Tyr Leu | 1190 |
| Ser Lys Ala Ala Ala Thr Tyr Val Gln Glu | 1200 |
| Phe Leu Pro His Ser Ile Cys Ala Met Gln | 1210 |

What is claimed is:

1. An isolated polypeptide comprising the amino acid sequence of SEQ ID NO: 2, and fragments thereof.

2. An isolated nucleic acid encoding the polypeptide of claim 1, and fragments thereof.

3. The isolated nucleic acid of claim 2, which comprises the nucleotide sequence of SEQ ID NO: 1.

4. The isolated nucleic acid of claim 3, wherein the fragments comprise the nucleotides 3192 to 3197 of SEQ ID NO: 1.

5. An expression vector comprising the nucleic acid of any one of claims 2 to 4.

6. A host cell transformed with the expression vector of claim 5.

7. A method for producing the polypeptide of claim 1, which comprises the steps of:
(1) culturing the host cell of claim 6 under a condition suitable for the expression of the polypeptide; and
(2) recovering the polypeptide from the host cell culture.

8. An antibody specifically binding to the polypeptide of Claim 1.

9. A method for diagnosing the diseases associated with the deficiency of human PHKA1 gene in a mammal which comprises detecting the nucleic acid of any one of claims 2 to 4 or the polypeptide of claim 1.

10. The method of claim 9, wherein the diseases are cancers.

11. The method of claim 10, wherein the cancers are lung cancers.

12. The method of claim 9, wherein the detection of the nucleic acid of any one claims 2 to 4 comprises the steps of:
(1) extracting total RNA from a sample obtained from the mammal;
(2) amplifying the RNA by reverse transcriptase-polymerase chain reaction (RT-PCR) with a pair of primers to obtain a cDNA sample comprising the nucleotides 3192 to 3197 of SEQ ID NO: 1; and
(3) detecting whether the cDNA sample is obtained.

13. The method of claim 12, wherein one of the primers has a sequence comprising the nucleotides of SEQ ID NO: 1 containing nucleotides 3192 to 3197, and the other has a sequence complementary to the nucleotides of SEQ ID NO: 1 at any other locations downstream of nucleotide 3197, or one of the primers has a sequence complementary to the nucleotides of SEQ ID NO: 1 containing nucleotides 3192 to 3197, and the other has a sequence comprising the nucleotides of SEQ ID NO: 1 at any other locations upstream of nucleotide 3192.

14. The method of claim 12, wherein one of the primers has a sequence comprising the nucleotides of SEQ ID NO: 1 upstream of nucleotide 3194 and the other has a sequence complementary to the nucleotides of SEQ ID NO: 1 downstream of nucleotide 3195, or one of the primers has a sequence complementary to the nucleotides of SEQ ID NO: 1 upstream of nucleotide 3194 and the other has a sequence comprising the nucleotides of SEQ ID NO: 1 downstream of nucleotide 3195.

15. The method of claim 14, wherein the cDNA sample amplified from SEQ ID NO: 1 is 39 bp shorter than the cDNA sample amplified from PHKA1.

16. The method of claim 12 further comprising the step of detecting the amount of the amplified cDNA sample.

17. The method of claim 9, wherein the detection of the nucleic acid of any one of claims 2 to 4 comprises the steps of:
(1) extracting the total RNA of a sample obtained from the mammal;
(2) amplifying the RNA by reverse transcriptase-polymerase chain reaction (RT-PCR) to obtain a cDNA sample;
(3) bringing the cDNA sample into contact with the nucleic acid of any one of claims 2 to 4; and
(4) detecting whether the cDNA sample hybridizes with the nucleic acid of any one of claims 2 to 4.

18. The method of claim 17 further comprising the step of detecting the amount of hybridized sample.

19. The method of claim 9, wherein the detection of the polypeptide of claim 1 comprises the steps of contacting the antibody of claim 8 with a protein sample obtained from the mammal, and detecting whether an antibody-polypeptide complex is formed.

20. The method of claim 19 further comprising the step of detecting the amount of the antibody-polypeptide complex.
